(19) United States
(12) Patent Application Publication
(10) Pub. No.: US 2005/0107596 A1
(43) Pub. Date: May 19, 2005

(54) Anti-alpha3(IV)nc1 monoclonal antibodies and animal model for human anti-glomerular basement membrane autoantibody disease

(76) Inventors: Michael P Madaio, Bryn Mawr, PA (US); Michael L. Gallo, British Columbia (CA)

Correspondence Address: Jane T Gunnison;Fish & Neave, 1251 Avenue of the Americas, New York, NY, 10020-1104, US

(21) Appl. No.: 10/475,764

(22) PCT Filed: Apr. 23, 2002
(86) PCT No.: PCT/US02/13063

Related U.S. Application Data
(60) Provisional application No. 60/285,860, filed on Apr. 23, 2001.

(51) Int. Cl. C12N 5/06; C07K 16/18

(52) U.S. Cl. 530/388.25; 435/326

(57) ABSTRACT

The present invention relates to methods of making a mouse model of human anti-GBM disease, to mice produced by such methods, and to human antibodies and antigen-binding portions thereof that specifically bind to α3(IV) NC1 collagen. The present invention also relates to compositions comprising the above antibodies or portions thereof and methods of using such compositions for diagnosis, and to nucleic acid molecules encoding the above antibodies or portions thereof. The present invention further relates to methods of isolating compounds/peptides that specifically binds anti-α3(IV) NC1 antibodies, pharmaceutical compositions comprising these compounds/peptides and methods of using such compositions for diagnosis and treatment.

### RELATED INFORMATION

This application claims priority to U.S. provisional application No. 60/285,860 entitled “ANTI-α3(IV) NC1 MONOCLONAL ANTIBODIES AND ANIMAL MODEL FOR HUMAN ANTI-GLOMERULAR BASEMENT MEMBRANE AUTOANTIBODY DISEASE”, filed Apr. 23, 2001, incorporated herein in its entirety by this reference.

### FIELD OF THE INVENTION

The invention relates to human monoclonal antibodies and antigen-binding portions thereof specific for the NC1 domain of the α3 strand of type IV collagen (hereinafter “α3(IV) NC1”) and methods and compositions comprising such monoclonal antibodies. The invention further relates to an animal model for human anti-glomerular basement membrane autoantibody disease (anti-GBM disease).

### BACKGROUND OF THE INVENTION

Anti-GBM disease is a human autoimmune disease mediated by the spontaneous production of antibodies against α3(IV) NC1 collagen (Kalluri et al., J. Biol. Chem. 266:24018-24024 (1991)). This typically results in linear deposition of antibodies within the glomerular basement membrane (GBM) leading to rapidly progressive glomerulonephritis. When associated with lung hemorrhage, it is termed Goodpasture syndrome (Couser, Am. J. Kidney Dis. 11:449-464 (1988)). Identification and isolation of α3(IV) NC1 collagen as the principal target of the human autoantibody response has provided the unique opportunity to study the major antigenic epitopes in this disease (Kalluri et al., Proc. Assoc. Am. Physicians 108:134-139 (1996); Hellmark et al., J. Biol. Chem. 274:25862-25868 (1999); Hellmark et al., Kidney Int. 55:936-944 (1999)). Most of the pathogenic antibodies derived from serum and kidney eluates of patients react with this protein (Kalluri et al., J. Am. Soc. Nephrol. 6:1178-1185 (1995)). Furthermore, following immunization of normal mice with α3(IV) NC1 collagen, the animals develop disease that closely resembles the human form of the disease (Kalluri et al., J. Clin. Invest. 100:2263-2275 (1997)). Investigation of these antibodies indicates that the antibody production is antigen driven and is consistent with the notion that the antigen is hidden with defective or inefficient peripheral tolerance to anti-α3(IV) NC1 autoantibody producing B cells. Nevertheless further evaluation of the molecular basis of the human autoimmune response has been limited by lack of access to disease-relevant immune cells and individual pathogenic antibodies.

The present invention provides fully human monoclonal antibodies specific for α3(IV) NC1 and methods and compositions comprising such monoclonal antibodies. The invention further provides an animal model for human anti-GBM disease.

### SUMMARY OF THE INVENTION

The present invention provides methods of producing an animal model for human anti-GBM disease comprising the steps of immunizing a non-human animal with α3(IV) NC1 and testing the animal for the production of antibodies that bind α3(IV) NC1 and for phenotypic characteristics of anti-GBM disease. In a preferred embodiment, the animal is a XenoMouse® animal genetically engineered to produce human IgG in response to antigenic challenge. In another preferred embodiment, the animal is a XenoMouse® animal genetically engineered to produce human IgG2 (γ₂k) in response to antigenic challenge. In one aspect of the invention, the animal is immunized with the NC1 domain of α3 strand of type IV collagen (α3(IV) NC1). In another aspect of the invention, the animal is immunized with bovine α3(IV) NC1. In yet another aspect of the invention, the animal is immunized with recombinantly produced human α3(IV) NC1. Recombinantly produced human α3(IV) NC1 may be expressed in a variety of cells, both prokaryotic and eukaryotic, e.g., E. coli, baculovirus, or human fetal 293 kidney cells.

The invention also provides methods of producing an animal model for human anti-GBM disease comprising the steps of passively immunizing the animal with a monoclonal antibody specific for α3(IV) NC1, and testing the animal for phenotypic characteristics of anti-GBM disease. In a preferred embodiment, the animal is a mouse. In a more preferred embodiment, the animal is a XenoMouse® animal. In a even more preferred embodiment, the animal is XenoMouse II® animal. Preferably, the monoclonal antibody is specific to an epitope bound by Mab F 1.1. More preferably, the monoclonal antibody is Mab F1.1.

The present invention also provides the animals produced by the above methods of making an animal disease model for human anti-GBM disease. Such animal models for human anti-GBM disease are useful for further investigation of anti-GBM disease in vivo as well as the testing of therapies to treat this disease.

The invention also provides methods for evaluating strategies and/or compounds for preventing or treating anti-GBM disease using the mouse model of human anti-GBM disease. This unique anti-GBM disease model is directly applicable to the human form of the disease and should provide the means for evaluating the human α3(IV) NC1 autoantibody response. In preferred embodiments, the mouse model is used to test specific therapies aimed at modulation of either B cells producing human autoantibodies or the human pathogenic antibodies themselves, in vivo, prior to trial in patients with the spontaneous form of the disease. In addition to its use for evaluating the efficacy of candidate compounds or other therapeutic interventions, the model can be used to investigate the etiology of the disease. This new model of anti-GBM disease therefore provides both the means and unique reagents to decipher further the molecular basis of the human anti-GBM autoantibody response.

The present invention provides antibodies or antigen-binding portions thereof that specifically bind α3(IV) NC1. In certain embodiments, the antibodies or antigen-binding portions are isolated and may be polyclonal or monoclonal. In preferred embodiments, the antibodies are human monoclonal antibodies. The present invention includes antibodies that comprise a human heavy chain and/or human light chain, the entire human variable region or any portion thereof, including individual CDRs of an antibody provided herein.

In some embodiments, the antibody comprises the heavy chain variable region amino acid sequence shown in SEQ ID NO: 2. In other embodiments, the antibody comprises a heavy chain comprising the CDR1, CDR2 and CDR3 shown in Table 2 (SEQ ID NO: 2). In another embodiment, the antibody heavy chain comprises a portion of the amino acid sequence shown in Table 2 (SEQ ID NO: 2) from CDR1 through CDR3. In other embodiments, any of the above-described antibodies further comprises a light chain comprising the amino acid sequence shown in SEQ ID NO: 4 (see also Table 3), CDR1 through CDR3 or a portion thereof or CDR1, CDR2 and CDR3 of the amino acid sequence of the light chain variable region sequence shown in Table 3 (SEQ ID NO: 4).

The antibodies or portion thereof of the invention may be an immunoglobulin G (IgG), an IgM, an IgE, an IgA or an IgD molecule. In a preferred embodiment, the human antibody is an IgG and is an IgG1, IgG2, IgG3 or IgG4 subtype. In another preferred embodiment, the human antibody is an IgG2 subtype. In a more preferred embodiment, the human antibody is Mab F1.1. In another embodiment, the antibody or antigen-binding portion thereof is derived from an Fab fragment, an F(ab′)₂ fragment, an Fv fragment, a single chain antibody or a chimeric antibody. In another embodiment, the antibody or antigen-binding portion thereof forms part of a fusion protein.

According to another object, the invention provides a human anti-α3(IV) NC1 antibody or antigen-binding portion thereof that is labeled or derivatized. In preferred embodiments, the labeled or derivatized antibody or portions thereof is used in diagnostic methods or in methods of screening for compounds/peptides that bind the antibody or portions thereof.

In another aspect, the invention provides polynucleotide molecules comprising sequences encoding the heavy and light chain immunoglobulin molecules of the invention or portions thereof, particularly nucleotide sequences encoding the heavy and light chain variable regions, contiguous heavy and light chain amino acid sequences from CDR1 through CDR3 and individual CDR's. In one embodiment, the invention provides vectors and host cells comprising the nucleic acid molecule(s). In another embodiment, the invention provides a method of recombinantly producing the heavy and/or light chain, the antigen-binding portions thereof or derivatives thereof, including production by an immortalized cell, synthetic means, recombinant expression, or phage display.

In another aspect, the invention provides an immortalized cell line, such as a hybridoma that produces human anti-α3(IV) NC1 monoclonal antibody.

In another aspect, the invention provides a method for identifying a compound/peptide that specifically binds a anti-α3(IV) NC1 antibody of the invention or fragments thereof. The screening method comprises the steps of providing an anti-α3(IV) NC1 antibody or fragment thereof, providing a test compound/peptide, incubating the antibody or fragment thereof with the test compound/peptide, and determining the ability of the test compound to bind the antibody or fragment thereof. In a preferred embodiment, the isolated compound/peptide inhibits the binding of the anti-α3(IV) NC1 antibody to α3(IV) NC1. This can be determined in a competition assay wherein both α3(IV) NC1 and the compound/peptide are incubated with the anti-α3(IV) NC1 antibody or fragment thereof. In one embodiment, the test compound/peptide is a member of a library of small molecules or peptides. In another embodiment, the peptide library is a phage-display library. Preferably, the library is derived from cDNA, genomic DNA, semi-synthetic or fully synthetic, semi-random or random nucleic acid sequences. In another preferred embodiment, the anti-α3(IV) NC1 antibody used in the screening is labeled or derivatized.

In another aspect, the present invention provides anti-idiotype (“anti-Id”) antibodies directed against human anti-GBM antibodies. In certain embodiments, the anti-Id antibodies or antigen-binding portions thereof are isolated and may be polyclonal or monoclonal. In preferred embodiments, the anti-Id antibodies are human monoclonal antibodies. In certain embodiments, the human anti-Id antibodies specifically bind anti-GBM antibody or fragments thereof isolated from patients with anti-GBM disease or from an animal model of anti-GBM disease of the current invention. In certain embodiments, said human anti-GBM antibody or fragment thereof is isolated from a XenoMouse® animal, e.g., a XenoMouse II® animal. In one embodiment, the anti-Id antibody specifically binds Mab F1.1.

In a related aspect, the present invention provides a method for producing said anti-Id antibody. Said method comprises the step of immunizing a non-human animal with an anti-GBM antibody. In certain embodiments, said method further comprises isolating antibody-producing cells from said animal. In preferred embodiments, said non-human animal is a mouse, more preferably a XenoMouse® mouse, e.g., a XenoMouse II® mouse.

In accordance with another aspect, the invention provides pharmaceutical compositions and kits comprising the anti-α3(IV) NC1 antibody-binding compounds/peptides identified by the screening methods of the current invention and a pharmaceutically acceptable carrier, or the anti-Id antibodies of the invention and a pharmaceutically acceptable carrier. In a preferred embodiment, the pharmaceutical composition or kit further comprises another component, such as an imaging reagent or therapeutic agent. In preferred embodiments, the pharmaceutical composition or kit is used in diagnostic or therapeutic methods.

Another aspect of the invention comprises diagnostic methods. In one embodiment, the invention provides a method for diagnosing the presence and/or location of anti-α3(IV) NC 1 antibody in a sample, comprising contacting the sample with a diagnostic agent. The diagnostic agent can be immobilized on a solid support or be in solution. In certain embodiments, the method uses purified α3(IV) NC1 as the diagnostic agent. In other embodiments, the method uses as the diagnostic agent a compound/peptide identified by the screening methods of the invention or an anti-Id antibody of the invention that specifically binds to anti-α3(IV) NC1 antibody. In a preferred embodiment, an anti-α3(IV) NC1 antibody (e.g., Mab F1.1) or an antigen-binding portion thereof of the current invention is used as a positive control. In a preferred embodiment, α3(IV) NC1 or the compound/peptide or anti-Id antibody is labeled. The diagnostic methods may be used in vivo or in vitro. In another embodiment, there is provided a diagnostic method that comprises determining whether said compound/peptide or anti-Id antibody inhibits or decreases the level of anti-α3(IV) NC1 antibody in a subject (and/or alleviate the symptoms of anti-GBM disease in a subject).

Another object of the invention comprises therapeutic methods of using the pharmaceutical compositions of the invention. In one embodiment, the therapeutic method comprises administering an effective amount of the composition to a subject in need thereof. In a preferred embodiment, the subject is suffering from anti-GBM disease. In a more preferred embodiment, the method inhibits or decreases the binding of anti-α3(IV) NC1 antibody to α3(IV) NC1. In another embodiment, the method is performed along with other therapies (e.g., antibody removal by plasmapheresis). In a still further embodiment, the compound/peptide or anti-Id antibody is labeled with a radiolabel, a drug conjugate, an immunotoxin or a toxin, or is a fusion protein comprising a toxic peptide.

In another aspect, the present invention provides methods, vectors and/or host cells comprising the appropriate nucleic acid molecule(s) for producing a peptide identified by the screening methods of the invention that specifically binds an anti-α3(IV) NC1 antibody, including production by an immortalized cell, synthetic means, recombinant expression or phage display.

Other features and advantages of the invention will be apparent from the following detailed description, and from the claims.

### BRIEF DESCRIPTION OF THE DRAWINGS

FIG. 1 Proliferative and crescentic glomerulonephritis in XenoMouse II® strains after immunization with various α3(IV) NC1 preparations. Panel A: Normal glomerulus from a non-immunized mouse, 400x. Panel B: A glomerulus from a XenoMouse II® animal given an α3(IV) NC1 preparation. The glomerulii were enlarged, hypercellular and had crescent formation (arrow), indicating proliferative glomerulonephritis, 400x.

FIG. 2 Linear staining (direct immunofluorescence) in an XenoMouse II® animal after immunization with α3(IV) NC1 collagen. Panel A: Normal glomerulii from a control mouse with no IgG staining (200x). Panel B: Linear staining for IgG deposited on the GBM with less intense TBM staining in the kidney section of a XenoMouse II® animal given an a3(IV) NC1 collagen preparation, 400x.

FIG. 3 Passive administration of Mab F1.1 causes anti-GBM disease in XenoMouse II® animals.

FIG. 4 Mab F1.1 binds E. coli and 293 mammalian cell expressed recombinant human α3(IV) NC1 antigen.

FIG. 5 XenoMouse II® animals produce anti-GBM antibodies which recognize α3(IV) NC1 collagen. Multiple clones were obtained from a XenoMouse II® mouse immunized with α3(IV) NC1. These clones recognized both bovine and E. coli derived human antigens by ELISA.

FIG. 6 Binding of Mab F1.1 to bovine α3(IV) NC1 or E. coli expressed recombinant human α3 (IV) NC1 by ELISA.

FIG. 7 Peptides derived from epitope mapping of α3(IV) (Bora et al., J. Biol. Chem. 275:6030-6037 (2000)) inhibit Mab F1.1 binding to α3(IV) NC1. Varying concentrations of either C2 (bottom curve) or C6 (top curve) were mixed and incubated with F1.1 prior to addition to α3 (IV) NC1, and Ab binding/inhibition was determined by ELISA.

### DETAILED DESCRIPTION OF THE INVENTION

Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

The term “polypeptide” encompasses native or artificial proteins, protein fragments and polypeptide analogs of a protein sequence. A polypeptide may be monomeric or polymeric. Preferred polypeptides in accordance with the invention comprise the human heavy chain immunoglobulin molecules and the human kappa light chain immunoglobulin molecules, as well as antibody molecules formed by combinations comprising the heavy chain immunoglobulin molecules with light chain immunoglobulin molecules, such as the K light chain immunoglobulin molecules, as well as fragments and analogs thereof.

The term “isolated protein” or “isolated polypeptide” is a protein or polypeptide that by virtue of its origin or source of derivation (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

A protein or polypeptide is “substantially pure,” “substantially homogeneous” or “substantially purified” when at least about 60 to 75% of a sample exhibits a single species of polypeptide. The polypeptide or protein may be monomeric or multimeric. A substantially pure polypeptide or protein will typically comprise about 50%, 60, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide fragment” as used herein refers to a polypeptide that has an amino-terminal and/or carboxy-terminal deletion, but where the remaining amino acid sequence is identical to the corresponding positions in the naturally-occurring sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long.

The term “polypeptide analog” as used herein refers to a polypeptide that is comprised of a segment of at least 25 amino acids that has substantial identity to a portion of an amino acid sequence and that specifically binds α3(IV) NC1 under suitable binding conditions. Typically, polypeptide analogs comprise a conservative amino acid substitution (or insertion or deletion) with respect to the naturally-occurring sequence. Analogs typically are at least 20 amino acids long, preferably at least 50 amino acids long or longer, and can often be as long as a full-length naturally-occurring polypeptide.

Non-peptide analogs are commonly used in the pharmaceutical industry as drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p.392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

An “immunoglobulin” is a tetrameric molecule. In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as K and X light chains. Heavy chains are classified as μ, Δ, γ, ζ, or ∈, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

An “antibody” refers to an intact immunoglobulin, or to an antigen-binding portion thereof that competes with the intact antibody for specific binding. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide. An Fab fragment is a monovalent fragment consisting of the VL, VH, CL and CH I domains; a F(ab′)₂ fragment is a bivalent fragment comprising two Fab fragments linked by a disulfide bridge at the hinge region; a Fd fragment consists of the VH and CH1 domains; an Fv fragment consists of the VL and VH domains of a single arm of an antibody; and a dAb fragment (Ward et al., Nature 341:544-546, 1989) consists of a VH domain. A single-chain antibody (scFv) is an antibody in which a VL and VH regions are paired to form a monovalent molecules via a synthetic linker that enables them to be made as a single protein chain (Bird et al., Science 242:423-426, 1988 and Huston et al., Proc. Natl. Acad. Sci. USA 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc.

Natl. Acad. Sci. USA 90:6444-6448, 1993, and Poljak, R. J., et al., Structure 2:1121-1123, 1994). One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a “bispecific” or “bifunctional” antibody has two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992).

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature. Examples of isolated antibodies include an anti-α3(IV) NC1 antibody that has been affinity purified using α3(IV) NC1, an anti-α3(IV) NC1 antibody that has been synthesized by a hybridoma or other cell line in vitro, and a human anti-α3(IV) NC1 antibody derived from a transgenic mouse.

The term “human antibody” includes all antibodies that have one or more variable and constant regions derived from human immunoglobulin sequences. These antibodies may be prepared in a variety of ways, as described below.

A humanized antibody is an antibody that is derived from a non-human species, in which certain amino acids in the framework and constant domains of the heavy and light chains have been mutated so as to avoid or abrogate an immune response in humans. Alternatively, a humanized antibody may be produced by fusing the constant domains from a human antibody to the variable domains of a non-human species. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies.

The term “K_{off}” refers to the off rate constant for dissociation of an antibody from the antibody/antigen complex.

The term “K_{d}” refers to the dissociation constant of a particular antibody-antigen interaction.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≦1 μM, preferably ≦100 nM and most preferably ≦10 nM.

Fragments or analogs of antibodies or immunoglobulin molecules can be readily prepared by those of ordinary skill in the art following the teachings of this specification. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991).

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ∈-N,N,N-trimethyllysine, ∈-N-acetyllysine, O-phosphoserine, N-acetylserine, N-formylmethionine, 3-methylhistidine, 5-hydroxylysine, s-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

Unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”. “Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. An example of “high stringency” or “highly stringent” conditions is a method of incubating a polynucleotide with another polynucleotide, wherein one polynucleotide may be affixed to a solid surface such as a membrane, in a hybridization buffer of 6×SSPE or SSC, 50% formamide, 5×Denhardt's reagent, 0.5% SDS, 100 μg/ml denatured, fragmented salmon sperm DNA at a hybridization temperature of 42° C. for 12-16 hours, followed by twice washing at 55° C. using a wash buffer of 1×SSC, 0.5% SDS. See also Sambrook et al., supra, pp. 9.50-9.55.

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program.

The term “corresponds to” is used herein to mean that a polynucleotide sequence is identical to all or a portion of a reference polynucleotide sequence, or that a polypeptide sequence is identical to a reference polypeptide sequence. In contrast, the term “complementary to” is used herein to mean that the complementary sequence is identical to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 98 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, even more preferably at least 98 percent sequence identity and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99% sequence identity. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein.

As used herein, the terms “label” or “labeled” refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, P-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

The term “agent” is used herein to denote a chemical compound, a mixture of chemical compounds, a biological macromolecule, or an extract made from biological materials.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

The term patient includes human and veterinary subjects.

Throughout this specification and claims, the word “comprise” or variations such as “comprises” or “comprising”, will be understood to imply the inclusion of a stated integer or group of integers but not the exclusion of any other integer or group of integers.

Human Antibodies and Humanization of Antibodies

Human antibodies avoid certain of the problems associated with antibodies that possess mouse or rat variable and/or constant regions. The presence of such mouse or rat derived proteins can lead to the rapid clearance of the antibodies or can lead to the generation of an immune response against the antibody by a patient. Fully human anti-α3(IV) NC1 antibodies are expected to minimize the immunogenic and allergic responses intrinsic to mouse or mouse-derivatized Mabs and thus to increase the efficacy and safety of the administered antibodies.

Methods of Producing Animal Disease Model, Antibodies, and Antibody-Producing Cell Lines

Immunization

In one embodiment of the instant invention, human antibodies and disease model for human anti-GBM disease are produced by immunizing a non-human animal comprising some or all of the human immunoglobulin locus with an α3(IV) NC1 antigen or immunogenic fragment thereof. In a preferred embodiment, the non-human animal is a XenoMouse™. In a more preferred embodiment, the non-human animal is a XenoMouse II™.

The XenoMouse™ is an engineered mouse strain that comprises large fragments of the human immunoglobulin loci and is deficient in mouse antibody production. See, e.g., Green et al. Nature Genetics 7:13-21 (1994) and U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000.

The XenoMouse™ strains were engineered with yeast artificial chromosomes (YACs) containing 245 kb and 190 kb-sized germline configuration fragments of the human heavy chain locus and kappa light chain locus, respectively, which contained core variable and constant region sequences. Id. The XenoMouse™ produces an adult-like human repertoire of fully human antibodies, and generates antigen-specific human monoclonal antibodies (Mabs).

A second generation XenoMouse™, XenoMouse II™, contains approximately 80% of the human antibody repertoire through introduction of megabase sized, germline configuration YAC fragments of the human heavy chain loci and kappa light chain loci. See Mendez et al. Nature Genetics 15:146-156 (1997), Green and Jakobovits J. Exp. Med. 188:483-495 (1998), and U.S. patent application Ser. No. 08/759,620, filed Dec. 3, 1996, the disclosures of which are hereby incorporated by reference. XenoMouse II™ produces only human immunoglobulins (Jakobovits et al., Proc. Nat'l. Acad. Sci. 90:2551-2555 (1992); Green et al., Nature Genetics 7:13-21(1994)). Of particular relevance, B cells from this strain undergo IgM to IgG isotype switching after appropriate stimulation (Green et al., Nature Genetics 7:13-21 (1994)). The mice developed anti-GBM disease after immunization with α3(IV) NC1 collagen preparations, including bovine α3(IV) NC1 dimers, E. coli expressed recombinant human α3(IV) NC1, baculovirus expressed recombinant human α3(IV) NC1, and human fetal 293-kidney cell expressed human α3(IV) NC1 collagen. In addition, a monoclonal autoantibody produced from an animal immunized with bovine α3(IV) NC1 collagen is pathogenic and causes disease on passive transfer.

In another embodiment, the non-human animal comprising human immunoglobulin gene loci are animals that have a “minilocus” of human immunoglobulins. In the minilocus approach, an exogenous Ig locus is mimicked through the inclusion of individual genes from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described, inter alia, in U.S. Pat. No. 5,545,807, 5,545,806, 5,625,825, 5,625,126, 5,633,425, 5,661,016, 5,770,429, 5,789,650, 5,814,318, 5,591,669, 5,612,205, 5,721,367, 5,789,215, and 5,643,763, hereby incorporated by reference.

An advantage of the minilocus approach is the rapidity with which constructs including portions of the Ig locus can be generated and introduced into animals. However, a potential disadvantage of the minilocus approach is that there may not be sufficient immunoglobulin diversity to support full B-cell development, such that there may be lower antibody production.

In another embodiment, the invention provides a method for making anti-α3(IV) NC1 antibodies from non-human, non-mouse animals by immunizing non-human transgenic animals that comprise human immunoglobulin loci. One may produce such animals using the methods described in U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000. The methods disclosed in these patents may be modified as described in U.S. Pat. No. 5,994,619. In a preferred embodiment, the non-human animals may be rats, sheep, pigs, goats, cattle or horses.

In order to produce an anti-α3(IV) NC1 antibody and/or an animal model for human anti-GBM disease, a non-human animal comprising some or all of the human immunoglobulin loci is immunized with an α3(IV) NC1 antigen or immunogenic fragment thereof. In one embodiment, the α3(IV) NC1 antigen is isolated and/or purified α3(IV) NC1. In another embodiment, the α3(IV) NC1 antigen is bovine or human α3(IV) NC1. In another embodiment, the α3(IV) NC1 antigen is a fragment of α3(IV) NC1. In another embodiment, the α3(IV) NC1 antigen is a fragment that comprises at least one epitope of α3(IV) NC1. In some embodiments, the animal is immunized with recombinantly produced human α3(IV) NC1 antigen or immunogenic fragment thereof. Recombinantly produced α3(IV) NC1 may be expressed in a variety of cells, both prokaryotic and eukaryotic, e.g., E. coli, baculovirus, or human fetal 293 kidney cells. In another embodiment, the α3(IV) NC1 antigen is a cell that expresses α3(IV) NC1.

Immunization of animals may be done by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane and U.S. Pat. No. 5,994,619. In a preferred embodiment, the α3(IV) NC1 antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

Production and Use of Animal Models for Human Anti-GBM Disease

After immunization of an animal with an α3(IV) NC1 antigen, the animals are tested for the production of antibodies that bind α3(IV) NC1 and for phenotypic characteristics of anti-GBM disease, such as, without limitation, linear IgG deposits along the GBM and TBM, proliferative crescentic glomerulonephritis, weaker staining for C3, elevated serum creatinine, and proteinuria. In an alternative aspect, the invention also provides methods of producing an animal model for human anti-GBM disease comprising the steps of passively immunizing the animal with a monoclonal antibody specific for α3(IV) NC1, and testing the animal for phenotypic characteristics of anti-GBM disease as above. In preferred embodiments of the above methods, immunized animals are compared with unimmunized animals and/or animals immunized with normal human IgG to facilitate the evaluation of anti-GBM disease state.

In preferred embodiments, the animal is a mouse. In a more preferred embodiment, the animal is a XenoMouse® mouse. In a even more preferred embodiment, the animal is a XenoMouse II® mouse. Preferably, the monoclonal antibody is specific to an epitope bound by Mab F1.1. More preferably, the monoclonal antibody is Mab F1.1.

The invention also provides the animals produced by the above methods. Such animal models for human anti-GBM disease are useful for further investigation of anti-GBM disease in vivo as well as the testing of therapies to treat this disease. In a preferred embodiment, the animal is a mouse. In a more preferred embodiment, the mouse is a XenoMouse® mouse, e.g., XenoMouse II® mouse.

The invention also provides methods for evaluating strategies and/or compounds for preventing or treating anti-GBM disease using the animal model of human anti-GBM disease. In a preferred embodiment, the animal model is a mouse model. In a more preferred embodiment, the mouse model is a XenoMouse® mouse, e.g., XenoMouse II® mouse. This unique anti-GBM disease model is directly applicable to the human form of the disease and should provide the means for evaluating the human α3(IV) NC1 autoantibody response. In preferred embodiments, the mouse model is used to test specific therapies aimed at modulation of either B cells producing human autoantibodies or the human pathogenic antibodies themselves, in vivo, prior to trial in patients with the spontaneous form of the disease. In addition to its use for evaluating the efficacy of candidate compounds or other therapeutic interventions, the model can be used to investigate the etiology of the disease.

Production of Antibodies and Antibody-Producing Cell Lines

After immunization of an animal with an α3(IV) NC1 antigen, antibodies and/or antibody-producing cells may be obtained from the animal. In one embodiment, anti-α3(IV) NC1 antibody-containing serum is obtained from the animal by bleeding or sacrificing the animal. The serum may be used as it is obtained from the animal, an immunoglobulin fraction may be obtained from the serum, or the anti-α3(IV) NC1 antibodies may be purified from the serum. It is well known to one of ordinary skill in the art that serum or immunoglobulins obtained in this manner will be polyclonal. The disadvantage in using polyclonal antibodies prepared from serum is that the amount of antibodies that can be obtained is limited and the polyclonal antibody has a heterogeneous array of properties.

In another embodiment, antibody-producing immortalized cells may be prepared from the immunized animal. After immunization, the animal is sacrificed and B cells from spleen or lymph nodes are immortalized according to any means well-known in the art, including but not limited to transformation, such as with EBV or fusion with an appropriate immortalized cell line, such as myeloma cells, as is well-known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using α3(IV) NC1, a portion thereof, or a cell expressing α3(IV) NC1. In a preferred embodiment, the initial screening is performed using an enzyme-linked immunoassay (ELISA) or a radioimmunoassay. In a more preferred embodiment, an ELISA is used for initial screening. An example of ELISA screening is provided in WO 00/37504, herein incorporated by reference.

Anti-α3(IV) NC1 antibody-producing hybridomas are selected, cloned and further screened for desirable characteristics, including robust hybridoma growth, high antibody production and desirable antibody characteristics, as discussed further below. Hybridomas may be expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, the immunized animal is a non-human animal that expresses human immunoglobulin genes and B cells from spleen or lymph nodes are fused to a myeloma derived from the same species as the non-human animal. In a more preferred embodiment, the immunized animal is a XenoMouse™ and the myeloma cell line is a non-secretory mouse myeloma. In a even more preferred embodiment, the immunized animal is a XenoMouse II™ and the myeloma cell line is a non-secretory mouse myeloma. In certain embodiments, the myeloma cell line is Sp2 mIL6. See, e.g., Example 3.

In one embodiment, hybridomas are produced that produce human anti-α3(IV) NC1 antibodies. In a preferred embodiment, the hybridomas are mouse hybridomas, as described above. In another preferred embodiment, the hybridomas are produced in a non-human, non-mouse species such as rats, sheep, pigs, goats, cattle or horses. In another embodiment, the hybridomas are human hybridomas, in which a human non-secretory myeloma is fused with a human cell expressing an anti-α3(IV) NC1 antibody.

Nucleic Acids, Vectors, Host Cells and Recombinant Methods of Making Antibodies Nucleic Acids

The present invention also encompasses nucleic acid molecules encoding human anti-α3(IV) NC1 antibodies. In one embodiment, the nucleic acid molecule encodes a heavy and/or light chain of an intact human anti-α3 (IV) NC1 immunoglobulin. In a preferred embodiment, a single nucleic acid molecule encodes a heavy chain of a human anti-α3(IV) NC1 immunoglobulin and another nucleic acid molecule encodes the light chain of a human anti-α3(IV) NC1 immunoglobulin. In a more preferred embodiment, the encoded immunoglobulin is a human IgG. The encoded light chain may be a λ chain or a κ chain. In an even more preferred embodiment, the encoded light chain is a κ chain.

In preferred embodiments, the nucleic acid molecule encoding the variable region of the heavy chain (VH) is derived from a human DP-70 VH gene. In various embodiments, the nucleic acid molecule encoding the VH contains no more than ten, no more than six or no more than three amino acid changes from the germline DP-70 amino acid sequence. In certain embodiments, the nucleic acid molecule encoding the VH contains at least one amino acid change compared to the germline sequence, wherein said amino acid change is identical to an amino acid change in the heavy chain of the Mab F1.1 antibody compared to germline DP-70 sequence. In other embodiments, the VH contains at least three amino acid changes compared to the germline sequence, wherein said at least three amino acid changes are identical to at least three amino acid changes in the VH of the Mab F1.1 antibody compared to the germline sequence.

In some embodiments, the nucleic acid molecule encoding the VH region further comprises a nucleotide sequence derived from a human JH5b₁ gene.

Table 1 lists the nucleic acid sequences, and the corresponding amino acid sequences they encode, of the Mab F1.1 antibody or portions thereof, as well as the primers used to clone Mab F1.1 cDNA.

TABLE 1
List of sequences for Mab F1.1 and primers for cloning

| SEQ ID NO: | SEQUENCE INFORMATION |
|---|---|
| 1 | Heavy Chain variable region (V_{H}) DNA sequence |
| 2 | Heavy Chain variable region (V_{H}) PROTEIN sequence |
| 3 | Light Chain variable region (V_{L}) DNA sequence |
| 4 | Light Chain variable region (V_{L}) PROTEIN sequence |
| 5 | VH4g primer |
| 6 | CG2a primer |
| 7 | vK2A primer |
| 8 | Ck1d primer |

In preferred embodiments, the nucleic acid molecule comprises a nucleotide sequence encoding the amino acid sequence of the VH of Mab F1.1 at least from CDR1 through CDR3 (residues 31-109) as shown in Table 2. In another embodiment, the nucleic acid molecule comprises a nucleotide sequence encoding the amino acid sequence shown in SEQ ID NO: 2. In another embodiment, the nucleic acid molecule comprises the nucleotide sequence shown in SEQ ID NO: 1. In another embodiment, the nucleic acid molecule comprises a nucleotide sequence encoding of one or more of the CDRs of the heavy chain of Mab F1.1 shown in Table 2.

TABLE 2

AMINO ACID SEQUENCE (SEQ ID NO: 2) OF
Mab F1.1 HEAVY CHAIN VARIABLE REGION
The complementarity determining regions 1-3 (CDR1, CDR2
and CDR3) are bolded and underlined.

```
QVQLL ESGPG LVKPS GTLSL TCTVS GGSIS STNWW TWVRQ SPGTG LEWIG
                                                                            CDR1
HIYHS GSTDY NPSLK SRVTI SIDKS KNQFS LKMTS VTAAD TAVYY CACAA
              CDR2
QYHWK GLDPW GHGTL VTVSS
    CDR3
```

In other embodiments, the above-described nucleic acid molecules can hybridize under highly stringent conditions, such as those described above, to any one of the nucleic acid sequences described above.

In another aspect, the invention includes a nucleic acid molecule encoding the variable region of a light chain of a human anti-α3(IV) NC1 antibody, wherein said nucleic acid molecule comprises a nucleotide sequence derived from a human DPK-12 Vκ gene. Said nucleic acid molecule may contain up to ten, up to six or up to three amino acid substitutions from the germline DPK-12 Vκ amino acid sequence. In certain embodiments, the nucleic acid molecule encodes at least three amino acid substitutions compared to the germline Vκ amino acid sequence, wherein said substitutions are identical to the substitutions found in the light chain V region of Mab F1.1 compared to germline. In further embodiments, any of the foregoing the nucleic acid molecules further comprises a nucleotide sequence derived from a human Jκ5 joining segment gene.

In preferred embodiments, the nucleic acid molecule comprises a nucleotide sequence encoding the amino acid sequence of the light chain variable region of Mab F1.1, at least from CDR1 through CDR3 (residues 9-84) shown in Table 3. In another embodiment, the nucleic acid molecule comprises a nucleotide sequence encoding the amino acid sequence shown in SEQ ID NO: 4. In another embodiment, the nucleic acid molecule comprises the nucleotide sequence shown in SEQ ID NO: 3. In another embodiment, the nucleic acid molecule comprises a nucleic acid sequence that encodes the amino acid sequence of one or more of the CDRs shown in Table 3.

TABLE 3

AMINO ACID SEQUENCE (SEQ ID NO: 4) OF
Mab F1.1 LIGHT CHAIN VARIABLE REGION
The complementarity determining regions 1-3 (CDR1, CDR2
and CDR3) are bolded and underlined.

```
AHAVS ISCMS SQSLL HSDGK TYLYW YLQKP GQPPQ LLIYE VSNRF SGVPD
                              CDR1 CDR2
RFSGS GSGTD FTLKI SRVEA EDVGI YYCMQ SIQLC MQSIQ LITFG QGTRL
                                                                    CDR3
EIKR
```

In another aspect, the invention includes a nucleic acid molecule encoding a VL that hybridizes under highly stringent conditions, such as those described above, to a nucleic acid sequence encoding any of the above-described amino acid sequences.

A nucleic acid molecule encoding either the entire heavy or entire light chain of a human anti-α3(IV) NC1 antibody or the variable regions thereof may be obtained from any source that produces an anti-α3(IV) NC1 antibody. In one embodiment of the invention, the nucleic acid molecules may be obtained from a hybridoma that expresses an anti-α3(IV) NC1 antibody. Methods of isolating mRNA encoding an antibody are well-known in the art. See, e.g., Sambrook et al. The mRNA may be used to produce cDNA for use in the polymerase chain reaction (PCR) or cDNA cloning of antibody genes. In a preferred embodiment, the nucleic acid molecule is derived from a hybridoma that has as one of its fusion partners a transgenic animal cell that expresses human immunoglobulin genes. In a more preferred embodiment, the fusion partner animal cell is derived from a Xenomouse™ animal. In an even more preferred embodiment, the fusion partner animal cell is derived from a Xenomouse II™ animal. In another embodiment, the hybridoma is derived from a non-human, non-mouse transgenic animal as described above.

In a preferred embodiment, the heavy chain of an anti-α3(IV) NC1 antibody may be constructed by fusing a nucleic acid molecule encoding the variable domain of a heavy chain with a constant domain of a heavy chain. Similarly, the light chain of an anti-α3(IV) NC1 antibody may be constructed by fusing a nucleic acid molecule encoding the variable domain of a light chain with a constant domain of a light chain. In a more preferred embodiment, the nucleic acid encoding the variable region of the heavy chain encodes the amino acid sequence of SEQ ID NO: 2, and the nucleic acid molecule encoding the variable region of the light chains encodes an amino acid sequence comprising the amino acid sequence of SEQ ID NO: 4.

In another embodiment, an anti-α3(IV) NC1 antibody-producing cell itself may be isolated from a non-human animal. In one embodiment, the antibody-producing cell may be derived from a transgenic animal that expresses human immunoglobulin genes and has been immunized with an α3(IV) NC1 antigen. The transgenic animal may be a mouse, such as a XenoMouse™ mouse (e.g. XenoMouse II® mouse), or another non-human transgenic animal. In another embodiment, the anti-α3(IV) NC1 antibody-producing cell is derived from a non-transgenic animal. In another embodiment, the anti-α3(IV) NC1 antibody-producing cell may be derived from a human patient with an autoimmune disease who produces anti-α3(IV) NC1 antibodies. The mRNA from the anti-α3(IV) NC1 antibody-producing cells may be isolated by standard techniques, amplified using PCR and screened using standard techniques to obtain nucleic acid molecules encoding anti-α3(IV) NC1 heavy and light chains.

In another embodiment, the nucleic acid molecules may be used to make vectors using methods known to those having ordinary skill in the art. See, e.g., Sambrook et al. and Ausubel et al. In one embodiment, the vectors may be plasmid or cosmid vectors. In another embodiment, the vectors may be viral vectors. Viral vectors include, without limitation, adenovirus, retrovirus, adeno-associated viruses and other picoma viruses, hepatitis virus and baculovirus. The vectors may also be bacteriophage including, without limitation, M13.

The nucleic acid molecules may be used to recombinantly express large quantities of anti-α3(IV) NC1 antibodies, as described below. The nucleic acid molecules may also be used to produce chimeric antibodies, single chain antibodies, immunoadhesins, diabodies, mutated antibodies and antibody derivatives, as described further below.

In one embodiment, the nucleic acid molecules encoding the variable region of the heavy (VH) and light (VL) chains are converted to full-length antibody genes. In one embodiment, such nucleic acid molecules are inserted into expression vectors already comprising sequences encoding heavy chain constant or light chain constant regions, respectively, such that the VH or VL segment is operatively linked to the CH or CL segment(s), respectively, within the vector. In another embodiment, the nucleic acid molecules encoding the VH and/or VL chains are converted into full-length antibody genes by linking the nucleic acid molecule encoding a VH chain to a nucleic acid molecule encoding a CH chain using standard molecular biological techniques. The same may be achieved using nucleic acid molecules encoding VL and CL chains. The sequences of human heavy and light chain constant region genes are known in the art. See, e.g., Kabat et al., Sequences of Proteins of Immunological Interest, 5th Ed., NIH Publ. No. 91-3242, 1991.

In another embodiment, the nucleic acid molecules of the invention may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of anti-α3(IV) NC1 antibodies. In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or a part of one or more of the CDRs.

Vectors

To express the antibodies, or antibody portions of the invention, DNAs encoding partial or full-length light and heavy chains, obtained as described above, are inserted into expression vectors such that the genes are operatively linked to transcriptional and translational control sequences. Expression vectors include plasmids, retroviruses, cosmids, YACs, EBV derived episomes, and the like. The antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector. In a preferred embodiment, both genes are inserted into the same expression vector. The antibody genes are inserted into the expression vector by standard methods (e.g., ligation of complementary restriction sites on the antibody gene fragment and vector, or blunt end ligation if no restriction sites are present).

A convenient vector is one that encodes a functionally complete human CH or CL immunoglobulin sequence, with appropriate restriction sites engineered so that any VH or VL sequence can be easily inserted and expressed, as described above. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The recombinant expression vector can also encode a signal peptide that facilitates secretion of the antibody chain from a host cell. The antibody chain gene may be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

Non-Hybridoma Host Cells and Methods of Recombinantly Producing Protein

Nucleic acid molecules encoding anti-α3(IV) NC1 antibodies and vectors comprising these antibodies can be used for transformation of a suitable mammalian host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455 (which patents are hereby incorporated herein by reference).

Mammalian cell lines available as hosts for expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines. Cell lines of particular preference are selected through determining which cell lines have high expression levels. Other cell lines that may be used are insect cell lines, such as Sf9 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

Transgenic Animals

Antibodies of the invention can also be produced transgenically through the generation of a mammal or plant that is transgenic for the immunoglobulin heavy and light chain sequences of interest and production of the antibody in a recoverable form therefrom. In connection with the transgenic production in mammals, antibodies can be produced in, and recovered from, the milk of goats, cows, or other mammals. See, e.g., U.S. Pat. Nos. 5,827,690, 5,756,687, 5,750,172, and 5,741,957. In one embodiment, non-human transgenic animals that comprise human immunoglobulin loci are immunized with α3(IV) NC1 or a portion thereof. One may produce such transgenic animals using methods described in U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000. In another embodiment, the transgenic animals may comprise a “minilocus” of human immunoglobulin genes. The methods disclosed above may be modified as described in, inter alia, U.S. Pat. No. 5,994,619. In a preferred embodiment, the non-human animals may be rats, sheep, pigs, goats, cattle or horses. In another embodiment, the transgenic animals comprise nucleic acid molecules encoding anti-α3(IV) NC1 antibodies. In a preferred embodiment, the transgenic animals comprise nucleic acid molecules encoding heavy and light chains specific for α3(IV) NC1. In another embodiment, the transgenic animals comprise nucleic acid molecules encoding a modified antibody such as a single-chain antibody, a chimeric antibody or a humanized antibody. The anti-α3(IV) NC1 antibodies may be made in any transgenic animal. In a preferred embodiment, the non-human animals are mice, rats, sheep, pigs, goats, cattle or horses.

Phage Display Libraries

Recombinant anti-α3(IV) NC1 human antibodies of the invention in addition to the anti-α3(IV) NC1 antibodies disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. There are commercially available kits for generating phage display libraries (e.g., the Pharmacia Recombinant Phage Antibody System, catalog no. 27-9400-01; and the Stratagene SurfZAP™ phage display kit, catalog no. 240612). There are also other methods and reagents that can be used in generating and screening antibody display libraries (see, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum. Antibod. Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc Acid Res 19:4133-4137; and Barbas et al. (1991) Proc. Natl. Acad. Sci. USA 88:7978-7982.

In a preferred embodiment, to isolate human anti-α3(IV) NC1 antibodies with the desired characteristics, a human anti-α3(IV) NC1 antibody as described herein is first used to select human heavy and light chain sequences having similar binding activity toward α3(IV) NC1, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552-554; and Griffiths et al., (1993) EMBO J 12:725-734. The scFv antibody libraries preferably are screened using human α3(IV) NC1 as the antigen.

Once initial human VL and VH segments are selected, “mix and match” experiments, in which different pairs of the initially selected VL and VH segments are screened for α3(IV) NC1 binding, are performed to select preferred VL/VH pair combinations. Additionally, to further improve the quality of the antibody, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to α3(IV) NC1.

Following screening and isolation of an anti-α3(IV) NC1 antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention, as described below. To express a recombinant human antibody isolated by screening of a combinatorial library, the DNA encoding the antibody is cloned into a recombinant expression vector and introduced into a mammalian host cells, as described above.

In another aspect of the instant invention, phage display libraries of F(ab′)2, scFv, cDNA, genomic DNA, or random DNA sequences are screened against a monoclonal anti-α3(IV) NC1 antibody of the invention for peptides that interacts with the anti-α3(IV) NC1 antibody with high affinity and specificity. A “random peptide” refers to a peptide oligomer comprising two or more amino acid monomers and constructed by a random or stochastic process, although a random peptide can be constructed based on a framework motif, such as α3(IV) NC1 collagen sequences. A “random peptide library” refers not only to a set of recombinant DNA vectors that encodes a set of random peptides, but also to the set of random peptides encoded by those vectors, as well as the phage particles containing those random peptides.

Class Switching

Another aspect of the instant invention is to provide a mechanism by which the class of an anti-α3(IV) NC1 antibody may be switched with another. In one aspect of the invention, a nucleic acid molecule encoding VL or VH is isolated using methods well-known in the art such that it does not include any nucleic acid sequences encoding CL or CH. The nucleic acid molecule encoding VL or VH are then operatively linked to a nucleic acid sequence encoding a CL or CH from a different class of immunoglobulin molecule. This may be achieved using a vector or nucleic acid molecule that comprises a CL or CH chain, as described above. For example, an anti-α3(IV) NC1 antibody that was originally IgM may be class switched to an IgG. Further, the class switching may be used to convert one IgG subclass to another, e.g., from IgG1 to IgG2.

Antibody Derivatives

One may use the nucleic acid molecules described above to generate antibody derivatives using techniques and methods known to one of ordinary skill in the art.

Mutated Antibodies

In another embodiment, the nucleic acid molecules, vectors and host cells may be used to make mutated anti-α3(IV) NC1 antibodies. The antibodies may be mutated in the variable domains of the heavy and/or light chains to alter a binding property of the antibody. For example, a mutation may be made in one or more of the CDR regions to increase or decrease the K_{d} of the antibody for α3(IV) NC1, to increase or decrease K_{off}, or to alter the binding specificity of the antibody. Techniques in site-directed mutagenesis are well-known in the art. See, e.g., Sambrook et al. and Ausubel et al., supra. In a preferred embodiment, mutations are made at an amino acid residue that is known to be changed compared to germline in a variable region of an anti-α3 (IV) NC1 antibody. In a more preferred embodiment, one or more mutations are made at an amino acid residue that is known to be changed compared to the germline in a variable region of one of the anti-α3(IV) NC1 antibodies of the invention. In another embodiment, the nucleic acid molecules are mutated in one or more of the framework regions. A mutation may be made in a framework region or constant domain to increase the half-life of the anti-α3(IV) NC1 antibody. See, e.g., U.S. application Ser. No. 09/375,924, filed Aug. 17, 1999, herein incorporated by reference. A mutation in a framework region or constant domain may also be made to alter the immunogenicity of the antibody, to provide a site for covalent or non-covalent binding to another molecule, or to alter such properties as complement fixation. Mutations may be made in each of the framework regions, the constant domain and the variable regions in a single mutated antibody. Alternatively, mutations may be made in only one of the framework regions, the variable regions or the constant domain in a single mutated antibody.

In one embodiment, there are no greater than ten amino acid changes in either the VH or VL regions of the mutated anti-α3(IV) NC1 antibody compared to the anti-α3(IV) NC1 antibody prior to mutation. In a more preferred embodiment, there is no more than five amino acid changes in either the VH or VL regions of the mutated anti-α3(IV) NC1 antibody, more preferably no more than three amino acid changes. In another embodiment, there are no more than fifteen amino acid changes in the constant domains, more preferably, no more than ten amino acid changes, even more preferably, no more than five amino acid changes.

Fusion Antibodies and Immunoadhesins

In another embodiment, a fusion antibody or immunoadhesin may be made which comprises all or a portion of an anti-α3(IV) NC1 antibody linked to another polypeptide. In a preferred embodiment, only the variable regions of the anti-α3(IV) NC1 antibody are linked to the polypeptide. In another preferred embodiment, the VH domain of an anti-α3(IV) NC1 antibody are linked to a first polypeptide, while the VL domain of an anti-α3(IV) NC1 antibody are linked to a second polypeptide that associates with the first polypeptide in a manner in which the VH and VL domains can interact with one another to form an antibody binding site. In another preferred embodiment, the VH domain is separated from the VL domain by a linker such that the VH and VL domains can interact with one another (see below under Single Chain Antibodies). The VH-linker-VL antibody is then linked to the polypeptide of interest. The fusion antibody is useful to directing a polypeptide to an α3(IV) NC1-expressing cell or tissue. The polypeptide may be a therapeutic agent, such as a toxin, growth factor or other regulatory protein, or may be a diagnostic agent, such as an enzyme that may be easily visualized, such as horseradish peroxidase. In addition, fusion antibodies can be created in which two (or more) single-chain antibodies are linked to one another. This is useful if one wants to create a divalent or polyvalent antibody on a single polypeptide chain, or if one wants to create a bispecific antibody. In one embodiment, the fusion antibody or immunoadhesin is prepared using the variable regions from Mab F1.1. In another embodiment, the fusion antibody or immunoadhesin is prepared using one or more CDR regions from an anti-α3(IV) NC 1 antibody, such as from Mab F1.1.

Single Chain Antibodies

To create a single chain antibody (scFv), the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554). The single chain antibody may be monovalent, if only a single VH and VL are used, bivalent, if two VH and VL are used, or polyvalent, if more than two VH and VL are used.

In one embodiment, the single chain antibody is prepared using one or more of the variable regions from Mab F1.1. In another embodiment, the single chain antibody is prepared using one or more CDR regions from said anti-α3(IV) NC1 antibody.

Kappabodies, Minibodies, Diabodies and Janusins

In another embodiment, other modified antibodies may be prepared using anti-α3(IV) NC1-encoding nucleic acid molecules. For instance, “Kappa bodies” (Ill et al., Protein Eng 10: 949-57 (1997)), “Minibodies” (Martin et al., EMBO J 13: 5303-9 (1994)), “Diabodies” (Holliger et al., PNAS USA 90: 6444-6448 (1993)), or “Janusins” (Traunecker et al., EMBO J 10: 3655-3659 (1991) and Traunecker et al. “Janusin: new molecular design for bispecific reagents” Int J Cancer Suppl 7:51-52 (1992)) may be prepared using standard molecular biological techniques following the teachings of the specification.

In one embodiment, the modified antibodies are prepared using one or more of the variable regions from Mab F1.1. In another embodiment, the modified antibody is prepared using one or more CDR regions from said anti-α3(IV) NC1 antibody.

Chimeric Antibodies

In another aspect, bispecific antibodies can be generated. In one embodiment, a chimeric antibody can be generated that binds specifically to α3(IV) NC1 through one binding domain and to a second molecule through a second binding domain. The chimeric antibody can be produced through recombinant molecular biological techniques, or may be physically conjugated together. In addition, a single chain antibody containing more than one VH and VL may be generated that binds specifically to α3(IV) NC1 and to another molecule. Such bispecific antibodies can be generated using techniques that are well known for example, Fanger et al., Immunol Methods 4: 72-81 (1994), Winter and Harris, Immunol Today 14:243-246 (1993), and Traunecker et al. Int. J. Cancer (Suppl.) 7: 51-52 (1992).

In one embodiment, the chimeric antibodies are prepared using one or more of the variable regions from Mab F1.1. In another embodiment, the chimeric antibody is prepared using one or more CDR regions from said anti-α3(IV) NC1 antibody.

Derivatized and Labeled Antibodies

An antibody or antibody portion of the invention can be derivatized or linked to another molecule (e.g., another peptide or protein). In general, the antibodies or portion thereof is derivatized such that the α3 (IV) NC1 binding is not affected adversely by the derivatization or labeling. Accordingly, the antibodies and antibody portions of the invention are intended to include both intact and modified forms of the human anti-α3(IV) NC1 antibodies described herein. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another type of derivatized antibody is a labeled antibody. Useful detection agents with which an antibody or antibody portion of the invention may be derivatized include fluorescent compounds, including fluorescein, fluorescein isothiocyanate, rhodamine, 5-dimethylamine-1-napthalenesulfonyl chloride, phycoerythrin, lanthanide phosphors and the like. An antibody may also be labeled with enzymes that are useful for detection, such as horseradish peroxidase, β-galactosidase, luciferase, alkaline phosphatase, glucose oxidase and the like. When an antibody is labeled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a colored reaction product, which is detectable. An antibody may also be labeled with biotin, and detected through indirect measurement of avidin or streptavidin binding. An antibody may also be labeled with a predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

An anti-α3(IV) NC1 antibody may also be labeled with a radiolabeled amino acid. The radio-labeled anti-α3(IV) NC1 antibody may be used diagnostically, for example, as a positive control for determining anti-α3(IV) NC1 antibody levels in a subject. Examples of labels for polypeptides include, but are not limited to, the following radioisotopes or radionuclides—³H, ¹⁴C, ¹⁵N, ³⁵S, ⁹⁰Y, ⁹⁹Tc, ¹¹¹In, ¹²⁵I, ¹³¹I.

An anti-α3(IV) NC1 antibody may also be derivatized with a chemical group such as polyethylene glycol (PEG), a methyl or ethyl group, or a carbohydrate group.

Characterization of Anti-α3(IV) NC1 Antibodies

Class and Subclass of Anti-α3(IV) NC1 Antibodies

The class and subclass of anti-α3(IV) NC1 antibodies may be determined by any method known in the art. In general, the class and subclass of an antibody may be determined using antibodies that are specific for a particular class and subclass of antibody. Such antibodies are available commercially. The class and subclass can be determined by ELISA, Western Blot as well as other techniques. Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

In one embodiment of the invention, the antibody is a polyclonal antibody. In another embodiment, the antibody is a monoclonal antibody. The antibody may be an IgG, an IgM, an IgE, an IgA or an IgD molecule. In a preferred embodiment, the antibody is an IgG and is an IgG1, IgG2, IgG3 or IgG4 subtype. In a more preferred embodiment, the anti-α3(IV) NC1 antibodies are subclass IgG2. In another preferred embodiment, the anti-α3(IV) NC1 antibodies are the same class and subclass as Mab F1.1.

Molecule Selectivity

In another aspect of the invention, the anti-α3(IV) NC1 antibodies demonstrate molecule selectivity. In one embodiment, the anti-α3(IV) NC1 antibody has a selectivity for α3(IV) NC1 that is at least 10, 20, 50 or 100 times greater than its selectivity for any other protein other than α3(IV) NC1. In a preferred embodiment, the anti-α3(IV) NC1 antibody does not exhibit any appreciable specific binding to any other protein other than α3(IV) NC1. One may determine the selectivity of the anti-α3(IV) NC1 antibody for α3(IV) NC1 using methods well known in the art following the teachings of the specification. For instance, one may determine the selectivity using Western blot, FACS, ELISA or RIA. In a preferred embodiment, one may determine the molecular selectivity using Western blot.

Half-Life of Anti-α3(IV) NC1 Antibodies

According to another object of the invention, the anti-α3(IV) NC1 antibody has a half-life of at least one day in vitro or in vivo. In a preferred embodiment, the antibody or portion thereof has a half-life of at least three days. In a more preferred embodiment, the antibody or portion thereof has a half-life of five days or longer. In another embodiment, the antibody or antigen-binding portion thereof is derivatized or modified such that it has a longer half-life. For instance, the anti-α3(IV) NC1 antibodies may be derivatized with PEG, carbohydrates or other moieties known to increase the half-life of serum proteins. One having ordinary skill in the art, following the teachings of the specification and using techniques well known in the art, would be able to make a derivatized anti-α3(IV) NC1 antibody having an increased half-life relative to the native anti-α3(IV) NC1 antibody In another preferred embodiment, the antibody may contain point mutations to increase serum half life, such as described in U.S. application Ser. No. 09/375,924, filed Aug. 17, 1999.

The antibody half life may be measured by any means known to one having ordinary skill in the art. For instance, the antibody half life may be measured by Western blot, ELISA or RIA over an appropriate period of time.

Identification of α3(IV) NC1 Epitopes Recognized by Anti-α3 (IV) NC1 Antibody

The invention also provides an anti-α3(IV) NC1 antibody that binds the same antigen or epitope as a human anti-α3(IV) NC1 antibody. Further, the invention provides an anti-α3(IV) NC1 antibody that cross-competes with a human anti-α3(IV) NC1 antibody. In a preferred embodiment, the human anti-α3(IV) NC1 antibody is Mab F1.1. In another preferred embodiment, the human anti-α3(IV) NC1 comprises one or more CDRs from Mab F1.1. In a preferred embodiment, the anti-α3(IV) NC1 antibody is another human antibody.

One may determine whether an anti-α3(IV) NC1 antibody binds to the same antigen using a variety of methods known in the art. For instance, one may determine whether a test anti-α3(IV) NC1 antibody binds to the same antigen by using an anti-α3(IV) NC1 antibody to capture an antigen that is known to bind to the anti-α3(IV) NC1 antibody, such as α3(IV) NC1, eluting the antigen from the antibody, and then determining whether the test antibody will bind to the eluted antigen. One may determine whether an antibody binds to the same epitope as an anti-α3(IV) NC1 antibody by binding the anti-α3(IV) NC1 antibody to α3(IV) NC1 under saturating conditions, and then measuring the ability of the test antibody to bind to α3(IV) NC1. If the test antibody is able to bind to the α3(IV) NC1 at the same time as the anti-α3(IV) NC1 antibody, then the test antibody binds to a different epitope as the anti-α3(IV) NC1 antibody. However, if the test antibody is not able to bind to the α3(IV) NC1 at the same time, then the test antibody binds to the same epitope as the human anti-α3(IV) NC1 antibody. This experiment may be performed using ELISA, RIA or surface plasmon resonance. In a preferred embodiment, the experiment is performed using surface plasmon resonance. In a more preferred embodiment, BlAcore is used. One may also determine whether an anti-α3(IV) NC1 antibody cross-competes with an anti-α3(IV) NC1 antibody. In a preferred embodiment, one may determine whether an anti-α3(IV) NC1 antibody cross-competes with another by using the same method that is used to measure whether the anti-α3(IV) NC1 antibody is able to bind to the same epitope as another anti-α3(IV) NC1 antibody.

Light and Heavy Chain Usage

The invention also provides an anti-α3(IV) NC1 antibody that comprises light chain variable sequences encoded by a humanVκ gene and a human Jκ gene. In the monoclonal antibody F1.1, the κ light chains utilize a human DPK-12 Vκ gene joined to a human Jκ5 gene.

In preferred embodiments, the light chain variable region of the anti-α3(IV) NC1 antibodies of the invention contains the same amino acid substitutions, relative to the germline DPK-12 gene amino acid sequence, as Mab F1.1. For example, in some embodiments, the light chain variable region of the anti-α3(IV) NC1 antibody may contain one or more of the amino acid substitutions relative to DPK-12 germline sequence that are present in Mab F1. 1. In this manner, one can mix and match different features of antibody binding in order to alter, e.g., the affinity of the antibody for α3(IV) NC1 or its dissociation rate from the antigen.

In another embodiment, the light chain variable region contains amino acid substitutions at the same positions as in the F1.1 monoclonal antibody, but uses different amino acids in those positions. Preferably the substitution is conservative relative to the amino acid present at that position in Mab F1.1. For example, if glutamate is present in Mab F1.1 at a particular position and the glutamate represents a substitution compared to germline, according to the present invention, one may conservatively substitute aspartate at that position. Similarly, if the amino acid substitution is serine, one may replace the serine with threonine.

In another preferred embodiment, the light chain comprises an amino acid sequence that is the same as the amino acid sequence of the VL of Mab F1.1. In another preferred embodiment, the light chain comprises the amino acid sequence of SEQ ID NO: 4. In another highly preferred embodiment, the light chain comprises amino acid sequences that are the same as the CDR regions of the light chain of Mab F1.1 shown in Table 3. In another preferred embodiment, the light chain comprises an amino acid sequence from at least one CDR region of the light chain of Mab F1.1.

In another embodiment, the antibody or portion thereof comprises a lambda light chain.

The present invention also provides an anti-α3(IV) NC1 antibody or portion thereof comprises a human heavy chain or a sequence derived from a human heavy chain. In one embodiment, the heavy chain amino acid sequence is derived from a human V_{H} DP-70 gene family. In a more preferred embodiment, the heavy chain comprises no more than eight amino acid changes from germline V_{H} DP-70, more preferably no more than six amino acid changes, and even more preferably no more than three amino acid changes.

In a preferred embodiment, the VH of the anti-α3(IV) NC1 antibody contains the same amino acid substitutions, relative to the germline amino acid sequence, as Mab F1.1. In another embodiment, the amino acid substitutions are made in the same position as those found in the VH of Mab F1. 1, but conservative amino acid substitutions are made rather than using the same amino acid.

In another preferred embodiment, the heavy chain comprises an amino acid sequence that is the same as the amino acid sequence of the VH of Mab F1.1. In another highly preferred embodiment, the heavy chain comprises amino acid sequences that are the same as the CDR regions of the heavy chain of the F1.1 monoclonal antibody shown in Table 2. In another preferred embodiment, the heavy chain comprises an amino acid sequence from at least one CDR region of the heavy chain of the F1.1 monoclonal antibody.

Screening for Peptides that Specifically Bind to Anti-α3(IV) NC1 Antibodies

In another aspect, the invention provides a method for identifying a compound/peptide that specifically binds a anti-α3(IV) NC1 antibody of the invention or fragments thereof. The screening method comprise the steps of providing an anti-α3(IV) NC1 antibody or fragment thereof, providing a test compound/peptide, incubating the antibody or fragment thereof with the test compound/peptide, and determining the ability of the test compound to bind the antibody or fragment thereof. In a preferred embodiment, the isolated compound/peptide inhibits the binding of the anti-α3(IV) NC1 antibody to α3(IV) NC1. This can be determined in a competition assay wherein both α3(IV) NC1 and the compound/peptide are incubated with the anti-α3(IV) NC1 antibody or fragment thereof. In one embodiment, the test compound/peptide is a member of a library of small molecules or peptides. In a preferred embodiment, the peptide library is a phage-display library. In preferred embodiments, the library is derived from cDNA, genomic DNA, semi-synthetic or fully synthetic, semi-random or random nucleic acid sequences. In another preferred embodiment, the anti-α3(IV) NC1 antibody used in the screening is labeled or derivatized (as described above). In a preferred embodiment, the anti-α3(IV) NC1 antibody used in the above method is Mab F1.1. In preferred embodiments, the compounds/peptides isolated according to the above method do not induce production of anti-GBM antibodies in a subject.

Production of Anti-Idiotype Antibodies Directed against Human Anti-GBM Antibodies

In another aspect, the present invention provides anti-idiotype (”anti-Id”) antibodies directed against human anti-GBM antibodies. In certain embodiments, the anti-Id antibodies or antigen-binding portions thereof are isolated and may be polyclonal or monoclonal. In preferred embodiments, the anti-Id antibodies are human monoclonal antibodies. In certain embodiments, the human anti-Id antibodies specifically bind anti-GBM antibody or fragments thereof isolated from patients with anti-GBM disease or from an animal model of anti-GBM disease of the current invention. In certain embodiments, said human anti-GBM antibody or fragment thereof is isolated from a XenoMouse® animal, e.g., XenoMouse II® animal. In one embodiment, the anti-Id antibody specifically binds Mab F1.1.

In a related aspect, the present invention provides a method for producing said anti-Id antibody. Said method comprises the step of immunizing a non-human animal with an anti-GBM antibody. In certain embodiments, said method further comprises isolating antibody-producing cells from said animal. In preferred embodiments, said non-human animal is a mouse, more preferably a XenoMouse® mouse, e.g., a XenoMouse II® mouse.

Pharmaceutical Compositions and Kits

In accordance with another aspect, the invention provides pharmaceutical compositions and kits comprising the anti-α3(IV) NC1 antibody-binding compounds/peptides identified by the screening methods of the current invention and a pharmaceutically acceptable carrier, or the anti-Id antibodies of the invention and a pharmaceutically acceptable carrier. In a preferred embodiment, the pharmaceutical composition or kit further comprises another component, such as an imaging reagent or therapeutic agent. In preferred embodiments, the pharmaceutical composition or kit is used in diagnostic or therapeutic methods.

In another aspect, the present invention provides methods, vectors and/or host cells comprising the appropriate nucleic acid molecule(s) for producing a peptide identified by the screening methods of the invention or the anti-Id antibodies of the invention that specifically binds an anti-α3(IV) NC1 antibody, including production by an immortalized cell, synthetic means, recombinant expression or phage display.

Diagnostic Methods of Use

Another aspect of the invention comprises diagnostic methods. In one embodiment, the invention provides a method for diagnosing the presence and/or location of anti-GBM antibodies in a biological sample of a subject, comprising contacting the sample with a diagnostic agent. The diagnostic agent can be immobilized on a solid support or be in solution. In certain embodiments, the diagnostic agent is purified α3(IV) NC1. In some embodiments, the method uses as the diagnostic agent a compound/peptide identified by the screening methods of the invention or an anti-Id antibody of the invention that specifically binds to anti-α3(IV) NC1 antibody). The diagnostic methods may be used in vivo or in vitro. In a preferred embodiment, α3(IV) NC1, the compound/peptide, or the anti-Id antibody is labeled, e.g., to facilitate detection of the location of anti-GBM antibodies in a biological sample. In another embodiment, there is provided a diagnostic method that comprises determining whether said compound/peptide or anti-Id antibody inhibits or decreases the level of anti-GBM antibodies in a subject and/or alleviate the symptoms of anti-GBM disease in a subject. In preferred embodiments, the subject is a human. In other preferred embodiments, the subject is an Old World primate such as a cynomologous monkey, a rhesus monkey, a chimpanzee, or an ape.

In a preferred embodiment, an anti-α3(IV) NC1 antibody (e.g., Mab F1.1) or an antigen-binding portion thereof of the current invention is used as a positive control for the detection of anti-GBM antibodies in a biological sample. In preferred embodiments, the anti-GBM antibodies and the anti-α3(IV) NC1 antibody (positive control) may be detected in a conventional immunoassay, including, without limitation, an ELISA, an RIA, FACS, tissue immunohistochemistry, Western blot or immunoprecipitation.

In one embodiment, the anti-α3(IV) NC1 antibody (positive control) is directly labeled with a detectable label. In another embodiment, the anti-α3(IV) NC1 antibody is unlabeled and a second antibody or other molecule that can bind the anti-α3(IV) NC1 antibody is labeled. As is well known to one of skill in the art, a second antibody is chosen that is able to specifically bind the specific species and class of the anti-α3(IV) NC1 antibody (positive control) and/or the anti-GBM antibodies from the sample. For example, if the anti-α3(IV) NC1 antibody is a human IgG, then the secondary antibody may be an anti-human-IgG. Other molecules that can bind to antibodies include, without limitation, Protein A and Protein G, both of which are available commercially, e.g., from Pierce Chemical Co.

Suitable labels for the antibody or secondary have been disclosed supra, and include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S or ³H. Similarly, α3(IV) NC1 can be labeled as described above when used as a detection agent for the location of anti-GBM antibodies in a sample.

In preferred embodiments, the anti-α3(IV) NC1 antibodies used in the above methods as positive control is Mab F1.1.

In an alternative embodiment, anti-GBM antibodies can be assayed in a biological sample by a competition immunoassay utilizing α3(IV) NC1 standards labeled with a detectable substance and an unlabeled anti-α3(IV) NC1 antibody. In this assay, the biological sample, the labeled α3(IV) NC1 standards and the anti-α3(IV) NC1 antibody are combined and the amount of labeled α3(IV) NC1 standard bound to the unlabeled anti-α3(IV) NC1 antibody is determined. The amount of anti-GBM antibodies in the biological sample is inversely proportional to the amount of labeled α3(IV) NC1 standard bound to the anti-α3(IV) NC1 antibody. In preferred embodiments, the unlabeled anti-α3 (IV) NC1 antibody is Mab F1.1.

Therapeutic Use of Using the Composition Comprising the Anti-α3(IV) NC1 Antibody-Binding Compounds/Peptides

Another object of the invention comprises therapeutic methods of using the composition comprising the anti-α3(IV) NC1 antibody-binding compounds/peptides or derivatives thereof identified by the screening methods of the invention, or the anti-Id antibodies of the invention. In one embodiment, the therapeutic method comprises administering an effective amount of the composition to a subject in need thereof. In a preferred embodiment, the subject is suffering from anti-GBM disease. In a more preferred embodiment, the method inhibits or decreases the binding of anti-α3(IV) NC1 antibody to α3(IV) NC1. The antibody or portion thereof may be administered from three times daily to once every six months, and may be administered via an intravenous, subcutaneous, intramuscular, parenteral or topical route. In another embodiment, the method is performed along with other therapies (e.g. antibody removal by plasmapheresis). In a still further embodiment, the compound/peptide or anti-Id antibody is labeled with a radiolabel, a drug conjugate, an immunotoxin or a toxin, or is a fusion protein comprising a toxic peptide.

The present invention is illustrated in the following examples. These examples are for purposes of illustration only and are not to be construed as restricting the invention in any manner.

### EXAMPLE 1

### Preparation of α3(IV) NC1 Collagen Antigens

Bovine α3(IV) NC1 dimers were isolated from bovine testis after digestion with bacterial collagenase, gel filtration and reverse-phase HDLC using previously described methods (Gunwar et al., J. Biol. Chem. 266:15318-15324 (1991); Kalluri et al., Proc. Nat'l. Acad. Sci. USA 91:6201-6205 (1994)). Recombinant human α3(IV) NC1 antigen was prepared as described (Neilson et al., J. Biol. Chem. 268:8402-8405 (1993); Meyers et al., Kidney Int. 53:402-407 (1998). Briefly, MC 1061 E. coli containing helper plasmid pDMI-1 and α3(IV) NC1 cDNA were grown up, and recombinant α3(IV)NC1 expression was induced using IPTG. After lysing the bacteria with 6 M guanidine HCl, the α3(IV) NC1, which contains 6 tandem histidine residues in its leader sequence, was affinity purified over a nickel resin column using a step gradient elution (25-250 mM imidazole; 8 M urea). Baculovirus expressed human α3(IV) NC1 collagen was a kind gift of Dr. Charles Pusey (Turner et al., The Journal of Biological Chemistry 269:17140-17145 (1994)). Eucaryotic recombinant human α3(IV) NC1 was expressed in human fetal 293 kidney cells (Netzer et al., J. Biol. Chem. 274:11267-11274 (1999)). The α3(IV) NC1 preparations were quantitated by SDS-PAGE and by BCA protein assay. The purity of the preparations was determined using anti-α3(IV) collagen chain-specific antibodies, ELISA and Western blot prior to use as described (Kalluri et al., J. Clin. Invest. 100:2263-2275 (1997)).

### EXAMPLE 2

### Generation of Anti-GBM Disease in a XenoMouse II® Animal Mediated by Human Antibodies that bind to α3(IV)NC1

Genetically engineered XenoMouse II® animal strains were produced as previously described (Green et al., Nature Genetics. 7:13-21 (1994)). XenoMouse® animals were immunized with 25 μg of α3(IV) NC1 antigen preparations (see Example 1) subcutaneously in complete Freud's adjuvant (CFA), followed by a 25 μg subcutaneous soluble booster injection 2-3 weeks later (Kalluri et al., J. Clin. Invest. 100:2263-2275 (1997); Jakobovits et al., Proc. Nat'l. Acad. Sci. 90:2551-2555 (1992); Green et al., Nature Genetics 7:13-21 (1994)). A soluble boost of antigen (25 μg) was also given subcutaneously four days prior to sacrifice. Control XenoMouse animals were injected with either CFA alone (2 animals) or with normal human IgG in CFA (2 animals) and then were boosted with phosphate buffered saline according to the schedule described above. An additional four control mice were immunized with recombinant human α1(IV) NC1 produced in 293 cells as detailed above for α3(IV) NC1. Disease severity in the immunized mice was evaluated by measuring serum antibodies and creatinine levels, and urine protein excretion.

For evaluation of proteinuria, mice were placed for 24 hours in metabolic cages immediately prior to sacrifice. Proteinuria was measured using a BCA protein assay according to the manufacturer's instructions (Pierce, Rockford, Ill.). Specimens were tested in duplicate and measured against a standard curve. The specimens were diluted to fit the linear part of the standard curve.

Blood, kidney and lung tissues were collected from the mice at the time of sacrifice, and the tissues were either immediately snap-frozen in isopentane or fixed in 10% phosphate saline-buffered formalin. The lungs were expanded with OTC (1:1 dilution with PBS) prior to their removal. At sacrifice, serum samples were obtained for determination of serum creatinine concentrations (Sigma Chemical Co., St. Louis, Mo.) and anti-α3(IV) NC1 antibody levels. For histologic analysis, tissue fixed in 10% buffered formalin was sectioned, stained with both hematoxylin and eosin (H&E) or periodic acid Schiff (PAS), and visualized by light microscopy. The sections were coded and viewed in a blinded fashion. All sections were viewed in at least 10 fields without the examiner knowing the identity of the mouse. Frozen tissues were cryostat-sectioned (4μ) and stained with either FITC-conjugated goat anti-human IgG (Southern Biotechnology Associates, Inc., Birmingham Ala.), or goat anti-mouse C3 (Sigma Chemical Co., St. Louis, Mo.) as described (Green et al., Nature Genetics 7:13-21 (1994)).

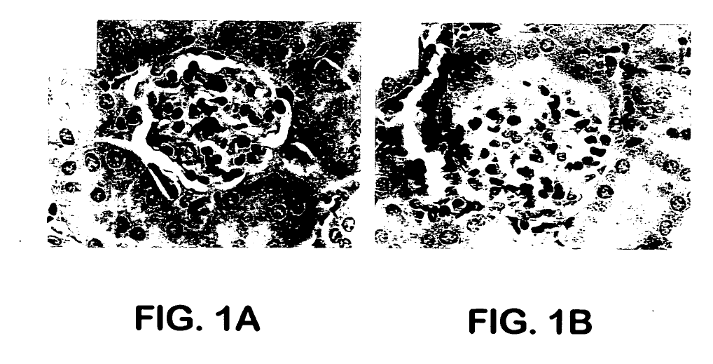

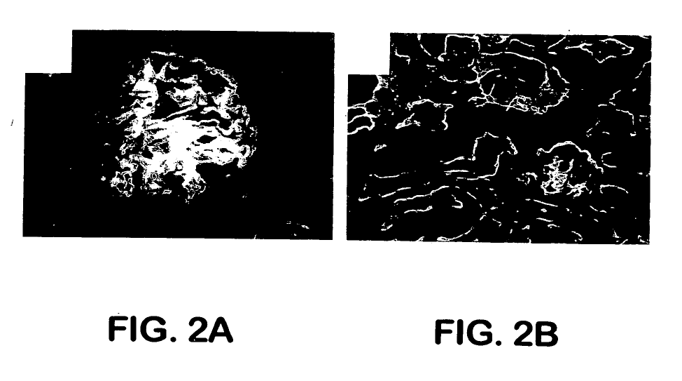

All XenoMouse II® animals developed human anti-GBM antibodies in serum following immunization with each of the α3(IV) NC1 collagen proteins (Table 4; see also FIGS. 1 and 2). By contrast neither unimmunized XenoMouse II® animals nor XenoMouse II® animals immunized with human IgG had detectable anti-α3(IV) NC1 antibody activity at any time (p<0.001) (FIGS. 1 and 2). All of the mice immunized with α3(IV) NC1 developed proteinuria, elevated serum creatinine levels and histologic evidence of nephritis. Proteinuria was present following immunization with each of the α3(IV) NC1 collagen preparations, the mean value three weeks after initial immunization varied to some extent with the immunogen (Table 4). The mean serum creatinine concentrations increased in all XenoMouse II® animals immunized with α3(IV) NC1 collagen (Table 1).

TABLE 4
Anti-GBM nephritis in XenoMouse II ® animals

| | | | Pr | | Prolifer- | Linear |
|---|---|---|---|---|---|---|
| Ag (no.) | Pre^{a} | Post^{b} | (mg/24 hr) | Cr (mg/dl) | ative GN | IF |
| Control# | 0.06 | 0.06 | <1.0 | 0.07 ± 0.03 | — | — |
| (4) | | | | | | |
| E. coli (4) | 0.09 | 2.8 | 2.4 | 0.4 | ++ | +++ |
| Bovine (4) | 0.14 | 0.62* | 2.8 | 1.03 | +++ | +++ |
| 293 Fetal | 0.14 | 2.3 | 1.6 | 1.1 | +++ | +++ |
| kidney(4) | | | | | | |

#Control XenoMouse II ® animals, 2 immunized with PBS and 2 immunized with normal human IgG
^{a}Average serum (1:100) anti-GBM antibody binding at OD 405 after the initial immunization with antigen (ELISA).
^{b}Average serum (1:100) anti-GBM antibody binding at OD 405 after the final immunization with antigen (ELISA). *After the second immunization.

Furthermore, all of the α3(IV) NC1 immunized XenoMouse® animals developed Proliferative glomerulonephritis (Table 4). By direct immunofluorescence all of the α3(IV) NC1 immunized XenoMouse® animals had linear IgG deposits along the GBM with variable and weaker TBM staining (Table 4). Murine C3 was also present, although the intensity of staining varied from 1-2+. Light microscopic evaluation of the lungs showed patchy alveolar hemorrhage in animals from all groups.

### EXAMPLE 3

### Hybridoma and Monoclonal Antibody Production

XenoMouse II® animals were immunized as in Example 2, with various forms of α3(IV) NC1, including native bovine, E. coli expressed and mammalian cell expressed antigen. We found that initial immunization in complete Freund's adjuvant, followed by booster injections of antigen in incomplete Freund's adjuvant leads to high serum antibody titers and severe nephritis. Disease severity in the immunized mice was evaluated by measuring serum antibodies and creatinine levels, and urine protein excretion. In the week prior to sacrifice, in anticipation of production of B cell hybridomas, the mice received daily doses of soluble antigen i.v. and/or i.p..

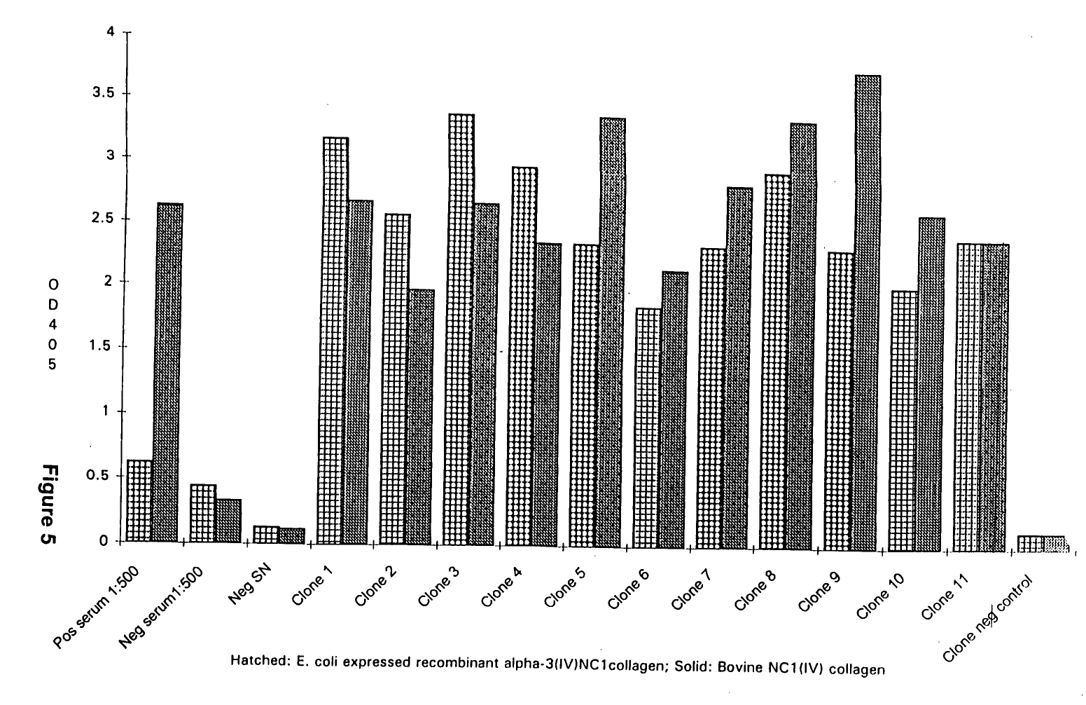

Immediately after sacrifice (approximately day 28), XenoMouse II® animal splenocytes were harvested and single cell suspensions prepared. B cell hybridomas were produced by fusing single spleen cell suspensions to the myeloma fusion partner Sp2 mIL6, as previously described (Lin et al., Hybridoma 18:257-261 (1999)). To select anti-α3(IV) NC1 antibody producing clones, ELISA was used to test supernatants from individual microtiter wells containing hybridomas (as assessed visually) for anti-α3(IV) NC1 antibody activity (Kalluri et al., Proc. Nat'l. Acad. Sci. USA 91:6201-6205 (1994)). Briefly, Immulon II plates were coated with 200 ng of either recombinant α3(IV) NC1 antigen or 100 ng of α3(IV) NC1 bovine collagen as previously described (Kalluri et al., J. Am. Soc. Nephrol. 6:1178-1185 (1995)). Multiple clones were obtained that produce anti-α3(IV) NC1 antibodies (FIG. 5). The titers of human anti-α3(IV) NC1 collagen Ab in the supernatant were then assayed by ELISA (Meyers et al., Kidney Int. 53:402-407 (1998)). Positive wells were subcloned (repeatedly), and the subclones were further tested for autoAb production. Subclones producing anti-α3(IV) NC1 Ab were frozen down, and the monoclonal antibodies were further evaluated and characterized (see Examples 4, 5, and 6 below).

To affinity purify anti-α3(IV) NC1 antibodies, an α3((IV)NC1 agarose column was prepared by coupling recombinant α3((IV)NC1 peptide to N-hydroxyssuccinimide activated Affigel 10 resin, where unreacted ester groups were blocked using 1 M ethanolamine (pH 8) prior to use. After transferring the gel to a column, which was previously washed extensively and equilibrated with PBS, anti-α3(IV) NC1 antibodies were isolated by passing small fractions (100-1000 ul) of hybridoma supernatant, diluted 1:5 in PBS, over the column. After extensive washing with PBS until free of reactants (assessed by taking a reading of the sample at OD₂₈₀), the antibodies were eluted in 1.5 ml fractions using 3 M NaSCN/PBS. The eluted antibodies were dialyzed in PBS and concentrated. Antibody concentration was determined (OD₂₈₀), and anti-α3(IV) NC1 antibody activity was assessed by ELISA. After repeated absorption, anti-α3(IV) NC1 antibody activity was not detectable in the effluents. (The same method was used to purify anti-GBM antibodies from patient serum.)

### EXAMPLE 4

### Characterization of Species of Origin and α3(IV) NC1-Binding Ability of Mab F1.1

Human gamma chain assay: Immulon II plates (Dynex Technologies, Chantilly Va.) were coated overnight at 4° C. with 500 ng of sheep anti-mouse IgG in borate buffer. The plates were then blocked with 1% bovine serum albumin (BSA). Undiluted supernatant from either XenoMouse II® animal derived Mab F1.1 and irrelevant mouse IgG Mab, or anti-GBM human serum (1:500) was added for 1 hour at 37° C. After washing, goat anti-mouse alkaline-phosphatase conjugated IgG, diluted 1/2000, was added for 1 hour at 37° C., and the plates were then developed as described (Meyers et al., Kidney Int. 53:402-407 (1998)).

Human kappa chain assay: Immulon II plates (Dynex Technologies, Chantilly Va.) were coated with 200 ng of E. coli expressed recombinant α3(IV) NC1 antigen as previously described. After blocking the plates with 1% BSA, supernatant from XenoMouse II® animal derived Mab F1.1 was added for 1 hour at 37° C. Biotinylated goat anti-human kappa antibody was added for 1 hour at 37° C. After washing, streptavidin conjugated to alkaline phosphatase, diluted 1/1000, was added for 1 hour at 37° C. The plates were then developed as described (Meyers et al., Kidney Int. 53:402-407 (1998)).

ELISA analysis of Mab F1.1 binding to α3(IV)NC1: Immulon II plates (Dynex Technologies, Chantilly Va.) were coated with 200 ng of either E. coli expressed recombinant α3(IV) NC1 antigen or 200 ng of human 293 fetal kidney cell expressed recombinant α3(IV) NC1 antigen in coating buffer. After blocking using 1% BSA/PBS and washing, monoclonal (e.g., Mab F1.1) or polyclonal anti-α3(IV)NC1 antibody preparations were added to the plates and incubated for 1 hour at 37° C. The plates were then washed and further incubated with 1:1000 alkaline phosphatase conjugated goat anti-human IgG for 1 hour at 37° C. The plates were then developed with p-nitrophenyl phosphate (Sigma, St. Louis Mo.) and read after 30 minutes at a wavelength of 405 nm.

Western Blot analysis of Mab F1.1 binding to α3(IV)NC1: Western blot analysis was carried out using a modified Lamely technique. A 12% SDS-Page running gel and 4% stacking gel were loaded with either 3 μg of recombinant E. coli expressed α3(IV) NC1 antigen or 5 μg of recombinant human α3(IV) NC1 expressed by 293 fetal kidney cells. After transfer onto Immobilon P membranes, blocking was carried out using 5% carnation milk/TBS Tween 0.05% (TBST) and incubated for an hour at 37° C. with monoclonal or polyclonal antibody preparations (polyclonal human sera were diluted 1:2000 (RP+, JG−)) and neat supernatants. The membrane lanes were then washed (1× with TBST, 1× with 0.05% NP40, 2× with TBST) and incubated for an hour at 37° C. with 1:2000 horseradish peroxidase-conjugated goat anti-human IgG. The membrane lanes were then rewashed and developed using the Enhanced Chemilumenescent System (ECS) according to the manufacturers instructions (Amersham, Buckinghamshire, England).

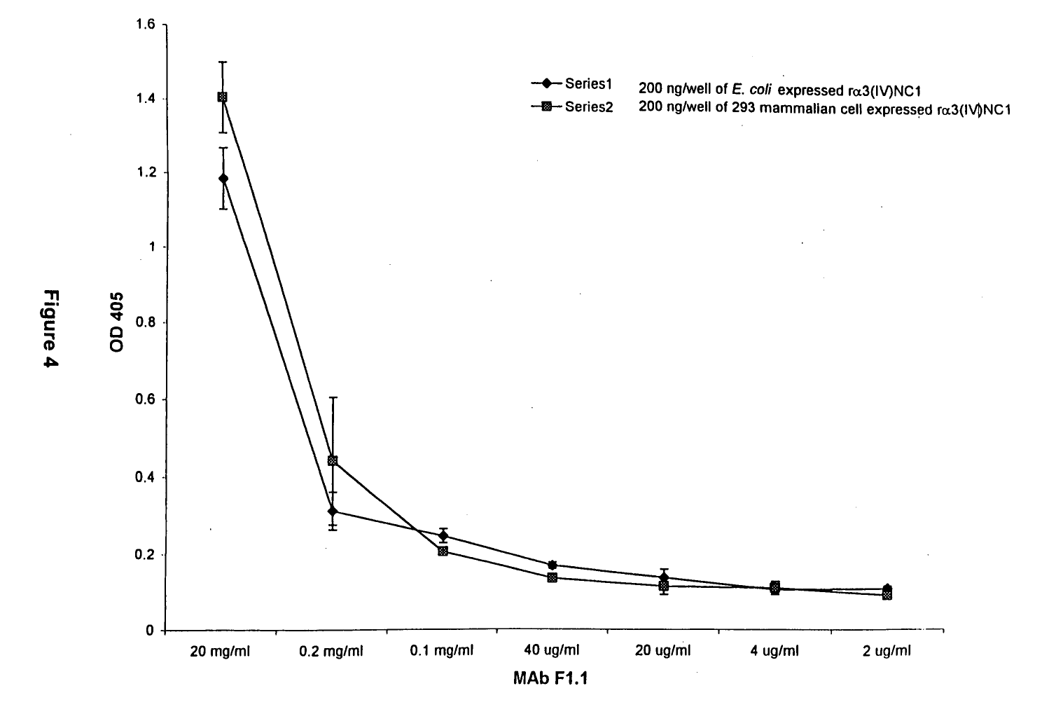

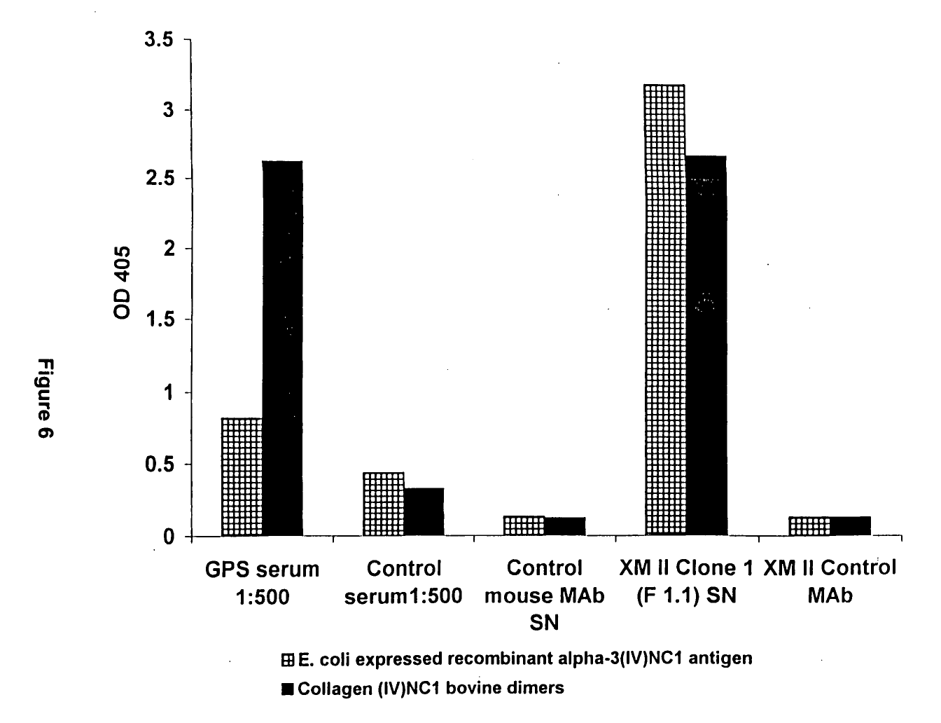

A fully human anti-GBM autoantibody that recognizes various α3(IV) NC1 collagen preparations: A monoclonal anti-α3(IV) NC1 antibody (MAb F1.1) derived from a XenoMouse® animal immunized with bovine NCI collagen was selected for further evaluation. The heavy chain assay and light chain assay indicated that the anti-α3(IV) NC1 collagen monoclonal autoantibody is fully human. It was necessary to establish this, as there can be a leak of mouse lambda light chain. This monoclonal Igγ₂k antibody bound bovine α3(IV) NC1 collagen, E. coli (prokaryote) and 293 cell (eukaryote)-expressed antigens by Western blotting and by ELISA (FIGS. 4 and 6). On solid phase ELISA testing, the Mab recognized bovine α3(IV) NC1 collagen, E. coli (prokaryocyte) and 293 cell (eukaryocyte)-expressed antigens well, although the intensity of the band with the 293 cell expressed antigen on Western blot was not as intense.

We deposited hybridoma F1.1 with the American Type Culture Collection (“ATCC”), 10801 University Boulevard, Manassas, Va. 20110-2209, USA, on Apr. 19, 2002. The F1.1 hybridoma has been given ATCC Accession Number ______.

### EXAMPLE 5

### Evaluation of Id-GBM Activity

Competitive inhibition assays were performed as previously described (Meyers et al., Kidney Int. 53:402-407 (1998)). Briefly, Immulon II wells were coated with 0.5 μg of recombinant α3(IV) NC1 affinity purified patient anti-GBM autoantibodies. Rabbit anti-Id serum (dilution predetermined by a direct binding titration assay) was then diluted in 3% BSAIPBS and pre-incubated with serial dilutions of XenoMouse II® animal derived monoclonal antibody (Id) supernatant for one hour at 37° C. The mixtures were then transferred into Immulon II wells and incubated for an additional one and a half hours at 37° C. After addition of alkaline phosphatase-conjugated goat anti-rabbit antibody (1:1000) that had been pre-adsorbed against human IgG the wells were incubated for an additional hour at 37° C. The wells were then washed, developed and read at wavelength 405 nm.

Mab F1.1 shared idiotype with anti-GBM antibodies derived from patients with active disease. We observed that all patients with anti-α3(IV) NC1 collagen antibody expressed this idiotype.

### EXAMPLE 6

### Passive Transfer Experiments: A Human Monoclonal Anti-GBM Antibody that Shares Structural Properties with Anti-GBM Autoantibodies Transfers Disease to Normal Mice

Four XenoMouse II® animals were intravenously administered 5 mg of purified Mab F1.1, a further four were injected intravenously with 5 mg affinity purified patient polyclonal anti-GBM autoantibody, and a further four control mice were injected intravenously with 5 mg normal human IgG. Five days after autoAb injection, quantitation of proteinuria was performed, the mice were sacrificed, and the kidneys were evaluated histologically as described above.

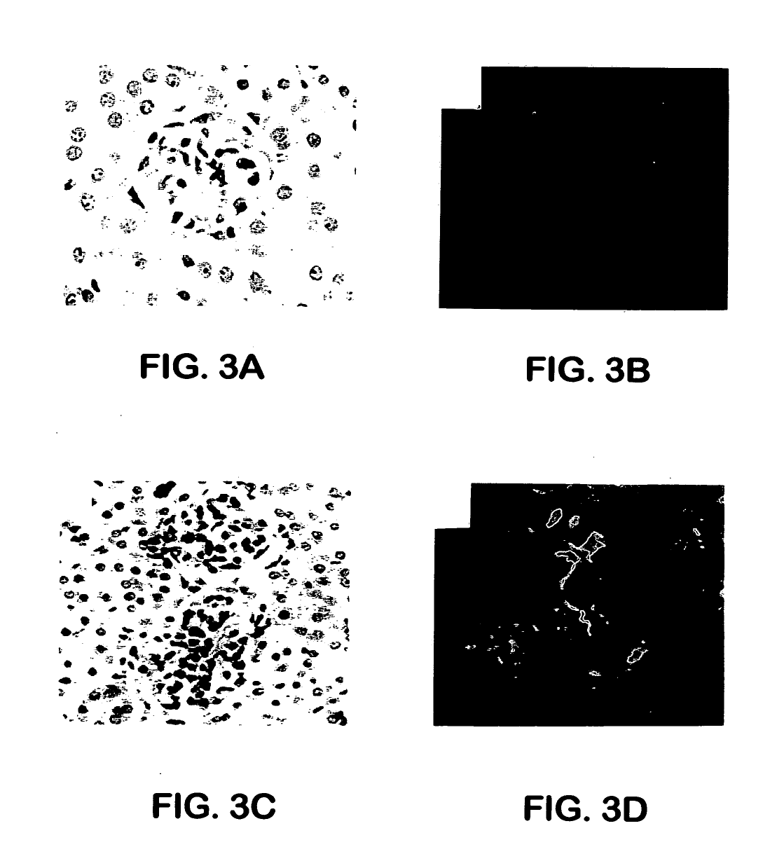

After IV administration of purified monoclonal or polyclonal Ab into XenoMouse II® animals, the animals developed proteinuria (Table 5), and a mild proliferative nephritis that was associated with human IgG deposition (FIG. 3). The direct IF staining was similar to the deposition observed after administration of polyclonal, affinity purified human anti-α3(IV) NC1 collagen Ab to XenoMouse II® animals. No proteinuria, nephritis or IF staining was noted after normal human IgG administration (FIG. 3). These results indicate that anti-GBM disease can be initiated in susceptible strains by human anti-α3(IV) NC1 antibodies, with an single specificity for α3(IV) NC1.

TABLE 5
Passive administration of Human anti-α3(IV)NC1 Mab F1.1

| Antigen | Urine protein* | Creatinine* | | |
|---|---|---|---|---|
| (no. mice) | mg/24 hrs | mg/dl | LM | Direct IF |
| Controls (4) | <1.0 | 0.07 ± 0.03 | Normal, no | No staining |
| | | | proliferation | |
| Monoclonal | 2.4 | 0.7 | Proliferative | Punctate 2+ |
| F1.1 (4) | | | GN 2+ | |
| Polyclonal | 1.6 | 0.8 | Patchy | Punctate 2+ |
| anti-GBM | | | mesangial | |
| Ab (4) | | | expansion | |

*Average urine protein and serum creatinine concentrations five days after passive antibody administration

### EXAMPLE 7

### Direct Immunofluorescence of XenoMouse II® Animal Kidneys

Mouse kidney tissue was frozen in OCT and sectioned to 4 μm. After fixing the tissue to positively charged slides (Fisher Scientific, Newark, Del.) they were washed three times with phosphate buffered saline, fixed for 10 minutes with ether/ethanol, and for 20 minutes with 95% ethanol. They were then rewashed three times in phosphate buffered saline and incubated with FITC-conjugated goat anti-human IgG. After a final wash step the slides were mounted (Aquamount, Fisher Scientific, Newark, Del.) and treated with anti-fade (Biorad, Hercules, Calif.). The slides were dried at 4° C. and viewed and imaged the following day.

As an example, direct immunofluorescence showed that all of the α3(IV) NC1 immunized XenoMouse® animals (see Example 2) had linear IgG deposits along the GBM with variable and weaker TBM staining. Murine C3 was also present, although the intensity of staining varied from 1-2+.

### EXAMPLE 8

### Binding of Monoclonal Anti-α3(IV) NC1 Autoantibody to Normal Human Kidney

Normal human kidney tissue was frozen in OCT and sectioned to 4 um. After fixing the tissue to positively charged slides (Fisher Scientific, Newark, Del.) they were washed three times with phosphate buffered saline, fixed for 10 minutes with ether/ethanol, and for 20 minutes with 95% ethanol. After rewashing with phosphate buffered saline the tissue was incubated with polyclonal anti-GBM serum or Mab F1.1 supernatant (20 mg/ml) diluted 1:50 for 1 hour. The tissue was then rewashed and incubated with FITC-conjugated goat anti-human IgG. After a final wash the tissue was mounted (Aquamount, Fisher Scientific, Newark, Del.) and treated with anti-fade (Biorad, Hercules, Calif.). The slides were dried at 4° C. and viewed and imaged the following day.

Mab F1.1 and control affinity purified patient (RP) anti-GBM autoantibodies bind in a linear fashion to normal human kidney by indirect immunofluorescence.

### EXAMPLE 9

### Structural Analysis of the Fully Human Anti-α3(IV) NC1 Monoclonal Antibodies

To analyze the structure of antibodies produced in accordance with the invention, we cloned and sequenced nucleic acids encoding at least a portion of the heavy and light chain from hybridomas producing human anti-α3(IV) NC1 monoclonal antibodies.

To clone cDNA encoding the variable regions of monoclonal antibody Mab F1.1, we isolated RNA from approximately 1×10⁶ hybridoma cells using QIAGEN™ RNeasy RNA isolation kit (QIAGEN). We reverse transcribed the mRNA using oligo-dT(18) and the Advantage™ RT/PCR kit (Clonetech). We used the following primers to amplify the cDNA.

| | |
|---|---|
| Primer name | Primer Sequence |

| | | |
|---|---|---|
| Heavy Chain | 5′- CAGGTGCAGCTACTCGAGTG | (SEQ ID NO: 5) |
| Sense: VH4g | GGG -3′ | |
| Heavy Chain | 5′- TTCTGTCAACTCGCGTTTTG | (SEQ ID NO: 6) |
| Anti-sense: | ATCACAGCTC -3′ | |
| CG2a | | |
| Light Chain | 5′- GATATTGAGCTCACTCAGTC | (SEQ ID NO: 7) |
| Sense: vK2A | TCCA -3′ | |
| Light Chain | 5′- GACTCAAGCGGGCAGTGTTT | (SEQ ID NO: 8) |
| Anti-sense: | CTCGAAGTTGTCCCCTCTCACAAT | |
| Ck1d | TAAGATCTGCCGCG -3′ | |

Mab cDNA light and heavy chain PCR products were agarose/TAE gels purified (with sephaglas beads, Amersham), cloned into TOPO vector (Invitrogen, Carlsbad, Calif.), transformed into chemically competent TOP10 One Shot® cells, and grown on LB agar/kanamycin plates. Individual colonies were picked, grown overnight and plasmid DNA was prepared. After restriction enzyme digestion, to control for the correct PCR inserts, the DNA was sequenced. The primers used for sequencing were standard T3 and T7 used for the Mab F1.1 heavy chain, and M 13 forward and reverse for the light chain. All sequencing reactions were performed using ABI 377 and 373A automated sequencers with Taq FS Big Dye™ Terminator or Dye Primer chemistry. For each clone, we verified the sequence on both strands in at least three reactions.

The determined heavy and light chain variable region DNA sequences and the deduced amino acid sequences of the F1.1 mAb are:

| | |
|---|---|
| CAGGTGCAGCTGCTCGAGTCGGGCCCAGGACTGGTG | (SEQ ID NO: 1) |
| AAGCCTTCGGGGACCCTGTCTCTCACCTGCACTGTC | |
| TCTGGTGGCTCCATCAGCAGTACTAACTGGTGGACT | |
| TGGGTCCGCCAGTCCCCAGGGACGGGTCTGGAGTGG | |
| ATTGGACATATCTATCATAGTGGGAGCACCGACTAC | |
| AACCCGTCCCTCAAGAGTCGAGTCACCATATCAATA | |
| GACAAGTCCAAGAATCAATTCTCCCTGAAGATGACC | |
| TCTGTGACCGCCGCGGACACGGCCGTCTATTACTGT | |
| GCGTGTGCGGCCCAGTATCACTGGAAGGGGCTCGAC | |
| CCCTGGGGCCATGGAACCCTGGTCACCGTCTCCTCA | |

Heavy Chain variable region (V_{H}) PROTEIN sequence

| | |
|---|---|
| QVQLLESGPGLVKPSGTLSLTCTVSGGSISSTNWWT | (SEQ ID NO: 2) |
| WVRQSPGTGLEWIGHIYHSGSTDYNPSLKSRVTISI | |
| DKSKNQFSLKMTSVTAADTAVYYCACAAQYHWKGLD | |
| PWGHGTLVTVSS | |

Light Chain variable region (V_{L}) DNA sequence

| | |
|---|---|
| GCTCACGCAGTCTCCATCTCCTGCATGTCTAGTCAG | (SEQ ID NO: 3) |
| AGCCTCCTTCATAGTGATGGAAAGACCTATTTGTAT | |
| TGGTACCTGCAGAAGCCAGGCCAGCCTCCACAGCTC | |
| CTGATCTATGAAGTTTCCAACCGGTTCTCTGGAGTG | |
| CCAGATAGGTTCAGTGGCAGCGGGTCAGGGACAGAT | |
| TTCACACTGAAAATCAGCCGGGTGGAGGCTGAGGAT | |
| GTTGGGATTTATTACTGCATGCAAAGTATACAGCTG | |
| TGCATGCAAAGTATACAGCTGATCACCTTCGGCCAA | |
| GGGACACGACTGGAGATTAAACGAA | |

Light Chain variable region (V_{L}) PROTEIN sequence

| | |
|---|---|
| AHAVSISCMSSQSLLHSDGKTYLYWYLQKPGQPPQL | (SEQ ID NO: 4) |
| LIYEVSNRFSGVPDRFSGSGSGTDFTLKISRVEAED | |
| VGIYYCMQSIQLCMQSIQLITFGQGTRLEIKR | |

Gene Utilization Analysis

By PCR primer extension analysis the Mab F1.1 belongs to the following VH and VL families: VH g2 and k2. The antibody HC and LC variable sequences were then compared and aligned using the V-Base germline databank (DNAPLOT 2.0. 1using V BASE Version 1.0).

Table 6 sets forth the gene utilization by the Mab F1.1 hybridoma in accordance with the invention.

TABLE 6
Heavy and Light Chain Gene Utilization

| Clone | Heavy Chain | | | Kappa Light Chain | |
|---|---|---|---|---|---|
| | VH | D | JH | VK | JK |
| F1.1 | DP-70 | | JH5b₁ | DPK-12 | JK5 |

Mutation Analysis

As will be appreciated, gene utilization analysis provides only a limited overview of antibody structure. As the B-cells in XenoMouse™ animals stochastically generate V-D-J heavy or V-J kappa light chain transcripts, there are a number of secondary processes that occur, including, without limitation, somatic hypermutation, additions, and CDR3 extensions. See, for example, Mendez et al., Nature Genetics 15:146-156 (1997) and International Patent Publication WO98/24893, published Oct. 11, 1998. The heavy chain CDR1 coding sequence contains at least two base pair changes and the CDR2 coding sequences contains three base pair changes compared with DP-70. The JH gene has 6 base pair changes compared with JH5b. The light chain CDR1 coding sequence contains two base pair changes from DPK-12, the CDR2 coding sequence is identical to genomic DPK-12, and the CDR3 coding sequence has the DPK-12/JK5 germline sequence.

To further examine antibody structure, we generated predicted amino acid sequences of the antibodies from the cDNAs obtained from the clones.

It will be appreciated that in addition to the above-identified amino acid substitutions or insertions, there exist substitutions within the FR's of Mab F1.1 from the germline. The CDR substitutions and FR substitutions in close proximity to a CDR would appear to bear some effect upon the binding of the antibody to the α3(IV) NC1 molecule. Further, such substitutions could have significant effect upon the affinity of the antibodies.

### EXAMPLE 10

### Screen for Peptides with High Affinity and Specificity for Human Anti-GBM Antibodies

To identify ligands with the highest affinity for pathogenic human anti-α3(IV) NC1 Ab, screening of peptide libraries is performed. To increase the likelihood of success, multiple peptide libraries, including those expressed by pIII vector (type 3; e.g. M13 KE) and pVIII vector (type 8+8 or 88 like f88-4) are utilized (Scott J K, Phage display. A laboratory manual pp. 1.4-2.13 (2001)). The M13KE provides 5 copies of closely opposed peptides at one tip, whereas a higher number of well separated copies (e.g., 100) are dispersed among 3000 of PVIII by f88-4. Use of multiple libraries ensures that a larger array of peptides are present (i.e. some peptides not present in individual libraries (Scott J K, Phage display. A laboratory manual pp. 1.4-2.13 (2001)). Peptide library based on the actual α3(IV) primary amino acid sequence can also be produced. Recombinant human α3(IV) cDNA is used as template to prepare a library through error-prone PCR or as described previously. This semi-random α3(IV) library is then screened as described above. Other phage display peptide libraries comprising cDNA, genomic, or random sequences can also be used in the screen.

Generally, several rounds of selection are performed to select the most interactive peptides, with each round followed by phage amplification. The amplification enhances the effectiveness of selection. To maximize affinity discrimination but also allow phage capture, lower stringency is used in early rounds of selection, with increasing stringency in latter rounds, so that the phages expressing peptides with highest affinity are captured early on and enriched as selection proceeds (Scott J K, Phage display. A laboratory manual pp. 1.4-2.13 (2001); and Scott J K & Smith G P, Science 249:386-390 (1990)).

To enhance the yield of interactive phage, biotinylated Mab F1.1 (or other monoclonal anti-α3(IV) Ab's, as well as affinity purified human anti-α3(IV) Ab's (i.e. derived from patient sera after elution from α3(IV) sepharose)) is employed. To decrease the yield of lower affinity peptides, biotinylated Mab F1.1 is immobilized in non-saturating amounts to strepavidin and then used for phage selection. Mab F1.1 is oriented on beads through Fc to minimize non-specific interactions.

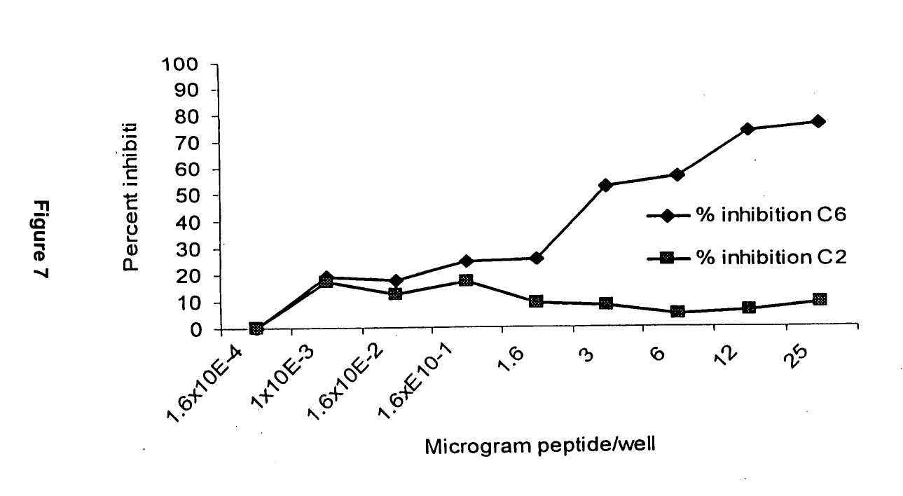

As basis for comparison of selected peptides, peptides derived from epitope mapping of α3(IV) (Bora et al., J. Biol. Chem. 275:6030-6037 (2000)) were tested for their ability to inhibit Mab F1.1 binding to α3(IV) NC1 (FIG. 7). Varying concentrations of either C2 (bottom curve) or C6 (top curve) were mixed and incubated with F1.1 prior to addition to α3(IV) NC1, and Ab binding/inhibition was determined by ELISA.

Once identified, the amino acid sequences of the peptides are determined (i.e. by determining the nucleotide sequences of the inserts of the phage, using appropriate primers) and the peptides synthesized. AutoAb binding to the synthetic peptides will be confirmed by direct binding (ELISA) and competitive inhibition, and the affinity of the individual peptides for human anti-α3(IV)Ab will be determined by both competitive inhibition assays and direct affinity measurements (BIAcore). In separate experiments, the capacity of the peptides to inhibit the binding to normal human kidney sections will also be evaluated.

The number of repeating units of the identified peptides for maximal inhibition of the anti-α3(IV) Ab-α3(IV) interaction will be optimized in vitro, prior to consideration for use in vivo (Kieber-Emmons et al., Curr. Opin. Biotechnol. 8:435-441 (1997)). The newly established XenoMouse II® animal model of human anti-α3(IV) Ab disease represents an excellent model to evaluate the use of therapies directed at either inhibition of either Ag-Ab interactions or at modulation of B cells producing pathogenic autoAb. Following optimization in vitro, the reagents derived from these studies are evaluated in vivo, during active disease in XenoMouse II® animals, to modulate autoAb production and deposition, prior to consideration for use in patients.

Potential carbohydrate inhibitors can be selected by screening random carbohydrate library screening (Kieber-Emmons, T. et al., J. Immunol. 165:623-627 (2000)).

### EXAMPLE 11

### Production of Anti-Idiotype Antibodies Directed Against Human Anti-GBM Antibodies

Anti-idiotype antibodies that specifically bind human anti-GBM antibodies were prepared and assayed as follows. Briefly, rabbits were immunized subcutaneously with 40 ug of affinity purified anti-GBM antibodies obtained from a single patient. Booster doses of 20-30 ug anti-GBM antibodies were given every 2 weeks for a total of four boosters. Serum was obtained from the rabbits, purified to remove irrelevant anti-human Ig activity, and tested for anti-idiotype activity as follows.

For purification of the anti-idiotype antibody from the hyperimmune sera, irrelevant rabbit anti-human Ig activity was removed using human IgG sepharose. For production of human IgG sepharose, normal human IgG was first passed over an α3(IV)NC1 Ag sepharose column to remove any non-specific binding; no IgG was eluted from the column indicating that anti-GBM antibodies are not normally present in human serum. The human IgG was then irreversibly cross-linked and oriented onto protein G using dimethylpimelimidate. Aliquots of hyperimmune rabbit serum were then passed over the IgG sepharose column to remove anti-human antibodies with non-specific binding activity. When rabbit anti-human Ig was no longer detectable in the serum sample, the effluent, was tested for anti-Id activity.

Several methods were used to evaluate the interaction of the rabbit anti-Id antibodies with anti-GBM antibodies. Multiple aliquots of rabbit anti-Id antibodies were purified with similar and specific anti-Id activity. Data from ELISA assys indicated that there was a two log fold greater binding of rabbit anti-Id antibodies to anti-GBM antibodies as compared with normal IgG. The specificity of the interactions were confirmed by competitive inhibition assays, where the rabbit anti-Id antibodies specifically inhibited the binding of the original preparation of anti-GBM antibodies to α3(IV)NC1. The inhibition was not due to rabbit anti-Id antibodies binding to the α3(IV)NC1, since direct binding of the anti-Id antibodies to α3(IV)NC1 was never observed. Lack of binding of the purified anti-Id antibodies to α3(IV)NC1 on Western blots also supported this conclusion.

To further evaluate the specificity of the interaction between rabbit anti-Id antibody and the anti-GBM antibody, Western blotting using reduced and non-reduced human anti-GBM antibodies, was performed. For this purpose, purified anti-GBM antibodies were run on 7.5% SDS PAGE gels under reducing and non-reducing conditions. The proteins were then transferred to Immobilon P, blocked, and then incubated with either rabbit IgG or purified rabbit anti-Id antibodies. The anti-Id antibodies bound to intact anti-GBM antibodies, but did not bind to either the individual heavy or light chains of the anti-GBM antibodies, indicating that the epitope(s) (or idiotope(s) recognized by rabbit anti-Id antibodies requires the presence of both heavy and light chains in assembled form.

To determine Id-GBM activity in different patients, a competitive inhibition assay was performed, where the capacity of rabbit anti-Id GBM antibodies to inhibit the binding of human anti-GBM antibodies to recombinant α3(IV)NC1 was quantitated. Significant inhibition by rabbit anti-Id GBM antibodies of human anti-GBM antibody binding to recombinant α3(IV)NC1 was observed. Rabbit anti-Id GBM antibodies also inhibited the binding of anti-GBM antibodies derived from all other patients examined, indicating that anti-GBM antibodies from different patients share common structures (as defined by the rabbit anti-Id antibody).

XenoMouse II® is an ideal strain for production of a human monoclonal anti-Id, because they express human Ig constitutively, and therefore irrelevant anti-human IgG activity should not be problematic. For anti-Id production, XenoMouse II® animal is immunized with an affinity purified human anti-α3(IV) Ab or a human anti-α3(IV)NC1 monoclonal antibody of the invention, e.g., Mab F1.1. Prior to use, the human serum autoAb is further purified on an anti-IgG2 column to remove other isotypes (so the mice do not produce anti-isotype specific Ab). Using this approach the mice should not produce irrelevant anti-human Ig Ab activity, because they express IgG2. This step is not necessary when Mab F1.1. or XenoMouse II® animal (IgG2) serum is used.

The capacity of the human anti-Id to inhibit the binding of Mab F1.1 (biotinylated) to α3(IV) NC1 is utilized for identifying human anti-Id GBM activity. Antibody-producing cells from mice with human anti-Id GBM activity can be immortalized and screened for anti-Id activity. Positive clones may be subcloned. Particular emphasis is devoted to identifying anti-Id that recognizes a conformational motif among anti-α3(IV) NC1 antibodies and inhibits binding to α3(IV) NC1. V gene sequence analysis of human monoclonal anti-Id is determined and such information can be used for conformational analysis to examine the anti-α3(IV) Ab-anti-Id interaction. This provides a complementary means for identifying potential peptide inhibitors, and it has the potential to provide additional information pertaining to the nature of the autoAb-α3(IV) interaction.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. An isolated human monoclonal antibody or an antigen-binding portion thereof that specifically binds α3(IV) NC1 collagen.

2. The antibody or an antigen-binding portion according to claim 1, wherein said antibody or antigen-binding portion competitively inhibits the binding of an anti-glomerular basement membrane (anti-GBM) auto-antibody from a patient with anti-GBM disease.

3. The antibody or an antigen-binding portion according to claim 1, wherein said antibody or antigen-binding portion, when administered to a mouse, causes human-like anti-GBM disease in said mouse.

4. The antibody or an antigen-binding portion according to any one of claims 1 to 3, wherein said antibody or antigen-binding portion is produced by immunizing a mouse capable of producing fully human antibodies with α3(IV) NC1 collagen.

5. The antibody or an antigen-binding portion according to claim 4, wherein said mouse is a XenoMouse mouse.

6. The antibody or an antigen-binding portion according to any one of claims 1 to 5, wherein said α3(IV) NC1 collagen is human.

7. A hybridoma cell line that produces the F1.1 monoclonal antibody, wherein said cell line has ATCC deposit No. PTA-4237.

8. The F1.1 monoclonal antibody produced by the cell line according to claim 7.

9. An antibody or an antigen-binding portion thereof comprising a heavy chain and a light chain, wherein the heavy chain amino acid sequence comprises the CDR1 through CDR3 amino acid sequence in SEQ ID NO: 2 and wherein the light chain amino acid sequence comprises the CDR1 through CDR3 amino acid sequence of SEQ ID NO: 4.

10. The antibody or an antigen-binding portion thereof according to claim 9, wherein said heavy chain amino acid sequence comprises the amino acid sequence in SEQ ID NO: 2 and wherein the light chain amino acid sequence comprises the amino acid sequence of SEQ ID NO: 4.

11. An antibody or an antigen-binding portion thereof comprising a heavy chain and a light chain, wherein the heavy chain is encoded by a nucleic acid comprising the nucleotide sequence of SEQ ID NO: 1 and wherein the light chain is encoded by a nucleic acid comprising the nucleotide sequence of SEQ ID NO: 3.

12. An antibody or an antigen-binding portion thereof comprising a heavy chain, wherein the heavy chain amino acid sequence comprises the amino acid sequence of SEQ ID NO: 2.

13. A nucleic acid comprising the nucleotide sequence of SEQ ID NO: 1.

14. An antibody or an antigen-binding portion thereof comprising a light chain, wherein the light chain amino acid sequence comprises the amino acid sequence of SEQ ID NO: 4.

15. A nucleic acid comprising the nucleotide sequence of SEQ ID NO: 3.

16. A host cell transformed with a the nucleic acid according to claim 13 or 15.

17. A The nucleic acid according to claim 13 or 15, operably linked to an expression control sequence.

18. A host cell transformed with a the nucleic acid according to claim 17.

19. An antibody or an antigen-binding portion thereof comprising a heavy chain, wherein the heavy chain comprises the amino acid sequences of the heavy chain CDR1, CDR2 and CDR3 shown in SEQ ID NOS: 6, 8, and 10, respectively.

20. An antibody or an antigen-binding portion thereof comprising a heavy chain, wherein the heavy chain utilizes the human VH gene, human D gene and human JH gene and the utilized by the antibody according to claim 8.

21. The antibody or an antigen-binding portion thereof according to claim 20, further comprising a kappa light chain, wherein said light chain utilizes the human Vκ gene and human Jκ gene utilized in the antibody according to claim 8.

22. A method for producing a mouse model for human anti-GBM disease, comprising the steps of:
a. providing a mouse capable of producing a fully human antibody; and
b. immunizing said mouse with α3(IV) NC1 collagen.

23. The method according to claim 22, wherein said mouse is a XenoMouse® mouse.

24. A method of inducing anti-GBM disease in a mouse, comprising administering to said mouse an antibody selected from the group consisting of: an antibody according to any one of claims 3 to 5 or 8 or an anti-α3(IV) NC1 collagen auto-antibody from a patient suffering from anti-GBM disease.

25. The method according to claim 22 or claim 24, wherein said α3(IV) NC1 collagen is selected from the group consisting of: baculovirus expressed recombinant α3(IV) NC1 collagen, bovine α3(IV) NC1 collagen dimers, E. coli expressed recombinant α3(IV) NC1collagen and human fetal 293-kidney cell expressed α3(IV) NC1 collagen.

26. The mouse produced by the method according to any one of claims 22 to 25.

27. A method for screening or identifying compositions for use in treating or preventing one or more symptoms of anti-GBM disease, comprising the steps of:
a. administering a composition to a mouse according to claim 26; and
b. detecting a decrease in said one or more symptoms.


SEQUENCE LISTING (8 sequences)

SEQ ID NO: 1 - DNA, 360 bp, Homo sapiens
caggtgcagctgctcgagtcgggcccaggactggtgaagccttcggggaccctgtctctcacctgcactgtctctggtggctccatcagcagtactaactggtggacttgggtccgccagtccccagggacgggtctggagtggattggacatatctatcatagtgggagcaccgactacaacccgtccctcaagagtcgagtcaccatatcaatagacaagtccaagaatcaattctccctgaagatgacctctgtgaccgccgcggacacggccgtctattactgtgcgtgtgcggcccagtatcactggaaggggctcgacccctggggccatggaaccctggtcaccgtctcctca

SEQ ID NO: 2 - protein, 120 aa, Homo sapiens
QVQLLESGPGLVKPSGTLSLTCTVSGGSISSTNWWTWVRQSPGTGLEWIGHIYHSGSTDYNPSLKSRVTISIDKSKNQFSLKMTSVTAADTAVYYCACAAQYHWKGLDPWGHGTLVTVSS

SEQ ID NO: 3 - DNA, 313 bp, Homo sapiens
gctcacgcagtctccatctcctgcatgtctagtcagagcctccttcatagtgatggaaagacctatttgtattggtacctgcagaagccaggccagcctccacagctcctgatctatgaagtttccaaccggttctctggagtgccagataggttcagtggcagcgggtcagggacagatttcacactgaaaatcagccgggtggaggctgaggatgttgggatttattactgcatgcaaagtatacagctgtgcatgcaaagtatacagctgatcaccttcggccaagggacacgactggagattaaacgaa

SEQ ID NO: 4 - protein, 104 aa, Homo sapiens
AHAVSISCMSSQSLLHSDGKTYLYWYLQKPGQPPQLLIYEVSNRFSGVPDRFSGSGSGTDFTLKISRVEAEDVGIYYCMQSIQLCMQSIQLITFGQGTRLEIKR

SEQ ID NO: 5 - DNA, 23 bp, Artificial Sequence
  Description of Artificial Sequence Primer
caggtgcagctactcgagtgggg

SEQ ID NO: 6 - DNA, 30 bp, Artificial Sequence
  Description of Artificial Sequence Primer
ttctgtcaactcgcgttttgatcacagctc

SEQ ID NO: 7 - DNA, 24 bp, Artificial Sequence
  Description of Artificial Sequence Primer
gatattgagctcactcagtctcca

SEQ ID NO: 8 - DNA, 58 bp, Artificial Sequence
  Description of Artificial Sequence Primer
gactcaagcgggcagtgtttctcgaagttgtcccctctcacaattaagatctgccgcg